(19) United States
(12) Patent Application Publication
(10) Pub. No.: US 2026/0115436 A1
(43) Pub. Date: Apr. 30, 2026

(54) METHODS AND SYSTEMS FOR TREATING HYDROCEPHALUS

(71) Applicant: MADISON SCIENTIFIC, INC., Chicago, IL (US)

(72) Inventors: Michael NAGY, Chicago, IL (US); Jeffrey NEUWIRTH, Chicago, IL (US); Alessandra Rivera HIDALGO, Chicago, IL (US)

(21) Appl. No.: 19/163,563

(22) PCT Filed: May 6, 2025
(86) PCT No.: PCT/US2025/027986

§ 371 Date: Sep. 9, 2025

Related U.S. Application Data
(60) Provisional application No. 63/643,071, filed on May 6, 2024.

(51) Int. Cl. A61M 27/00; A61M 39/00; A61M 39/24

(52) U.S. Cl. CPC A61M 27/006; A61M 39/24; A61M 2039/0009; A61M 2205/04; A61M 2205/18; A61M 2205/3303; A61M 2205/3327; A61M 2205/3334; A61M 2205/3368; A61M 2205/3553; A61M 2205/3592; A61M 2205/502; A61M 2205/581; A61M 2205/582; A61M 2205/583; A61M 2205/8206; A61M 2210/0687; A61M 2230/005

(57) ABSTRACT

A system for treating hydrocephalus in a patient including a valve assembly and an external device. The valve assembly includes a controller that actuates the valve assembly between an open position and a closed position. The valve assembly also includes one or more sensors operatively connected to the controller, where the one or more sensors generate internal sensor data indicating a condition of the patient, and the controller actuates the valve assembly based on the internal sensor data. The external device generates an audible or visual warning, alarm, or notification when an amount of charge in the battery is below a predetermined threshold, when the controller, the external device, or a remote server determine an event occurrence based on the internal sensor data, when a manual override of the valve assembly exceeds a predetermined duration, or when the external device attempts and fails to communicate with the valve assembly.

### CROSS REFERENCE TO RELATED APPLICATIONS

The application claims priority to U.S. Provisional No. 63/643,071, filed on May 6, 2024, entitled “Methods and Systems for Treating Hydrocephalus,” which is incorporated herein by reference.

### TECHNICAL FIELD

The present teachings relate to methods, systems, and components thereof for treating hydrocephalus, and more particularly to a cerebrospinal-fluid shunt valve system for treating hydrocephalus.

### BACKGROUND

Cerebrospinal Fluid (CSF) is a fluid surrounding the human brain that serves as a mechanical shock absorber and drains residual particles away from the brain. Structures in the brain produce CSF, and other structures drain it into the venous system. Overproducing and/or under draining CSF may cause a buildup of CSF in the head and spine, a condition known as hydrocephalus. Hydrocephalus can significantly increase pressure inside the cranium, leading to severe headaches, skull deformation, cognitive impairment, brain damage, or death. Shunts have traditionally been used to convey excess CSF to other parts of the human body. A typical shunt (e.g., a Ventriculoperitoneal shunt) includes a proximal catheter inserted into the brain's ventricles via a hole in the cranium, and a valve that opens when intracranial pressure (ICP) increases beyond a threshold to allow CSF to flow into a distal catheter attached to the valve. However, most conventional shunts fail within the first three years of implantation. A shunt failure can put a patient at risk of death or permanent cognitive injury, and usually requires revision surgery. Many shunt failures are attributed to obstruction of the fluid line due to CSF over drainage, triggered by instantaneous intracranial pressure (ICP) spikes corresponding to sudden posture changes, coughing, straining, shouting, etc.

Thus, it is desirable to have a system that shunts CSF from the cranium to another part of the body while preventing shunt failure due to over drainage or underdrainage.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure. This summary is intended to include various combinations of described aspects.

In accordance with an embodiment, a system for treating hydrocephalus includes an implant with a controller, and a software application (“app”) communicatively coupled to the controller. The app may be configured to send or receive data from the controller. In an embodiment, the data may include atmospheric data, such as an ambient pressure value, a temperature, and the like. In an embodiment, the app may be run on an external device, such as, for example, a tablet, a netbook, a smartphone, or a wearable device worn by the user (e.g., a smartwatch or other suitable device including a processor, a sensor, and a transceiver for transmitting sensor readings). In embodiments, the app may serve as a backup to the external device, for example, when the external device is not transmitting data to the controller, e.g., when the external device is lost, or broken, or otherwise unavailable.

In accordance with another embodiment, a system for treating hydrocephalus may include an implant and an external device communicatively coupled to the implant. The external device may include an input device operable to log event data, for example, information concerning an event where the implant patient is experiencing medical symptoms, e.g., headache, dizziness or light-headiness. Other event data may include exercise, exertion, eating, sleeping, bowel movements, etc. In an embodiment, the input device may be communicatively coupled to a global positioning system (GPS) device configured to determine a location of the implant patient during the event.

In accordance with another embodiment, a system for treating hydrocephalus may include an implant with a controller and a valve assembly, and an external device communicatively coupled to the implant. The external device may include mobile device such as, for example, a tablet, a netbook, or a smart phone. The external device may additionally or alternatively include a wearable device worn by the user (e.g., a smartwatch or other suitable example of a wearable device including a processor, a transceiver, and a sensor). In an embodiment, the external device may include at least one sensor for detecting atmospheric conditions, and an input device be operable to actuate a manual override mode. In an embodiment, the controller may execute an algorithm to control the valve assembly when the manual override mode is actuated. In an embodiment, it is contemplated that the input device may be operable to activate a mode to find the patient's wearable device when it is lost or missing. In an embodiment, activating the mode may cause the wearable device to alarm or send an audible notification therefrom to direct the patient to the wearable device.

In accordance with another embodiment, an implant may include a power supply configured to supply power to the implant. The power supply may embody a rechargeable battery that may be selectively discharged to a safe storage level for shipping or sterilization of the implant.

In accordance with another embodiment, a system for treating hydrocephalus includes an implant including a first catheter, a second catheter, a controller, and a valve assembly operatively coupled to the first and second catheters. The first catheter is configured to transfer cerebrospinal fluid (CSF) to the inlet of the valve assembly. The implant may include a pressure sensor configured to measure intracranial pressure (ICP) in an intracranial space, particularly a subarachnoid space within the cranium, or within parenchyma of a patient's brain. The implant may optionally include a sensor configured to detect an angular orientation of the implant patient's anatomy relative to gravity, i.e., a tilt angle. The pressure sensor and the tilt sensor may be respectively coupled to the controller to transmit the ICP and tilt angle readings to the controller. In an embodiment, the pressure sensor may be configured to measure ICP at a location outside fluid channels defined by the first and second catheters. In an embodiment, the first catheter may include a first lumen and a second lumen. The first lumen may define a fluid channel to drain CSF, and the second lumen may define a fluid column to communicate ICP. In an embodiment, the controller may detect a blockage event in at least one of the first and second lumens based on the ICP and tilt angle readings. In an embodiment, the controller may include logic to detect the blockage event when the angle of orientation indicates that the implant patient is in an upright orientation. In this embodiment, the controller may open the valve assembly and receive ICP data to determine the blockage event.

In accordance with another embodiment, a system for treating hydrocephalus may include an implant including a first catheter, a second catheter, a pressure sensor, a controller, and a valve operatively coupled with the first and second catheters. In an embodiment, the first catheter may include a lumen defining a fluid channel to drain CSF, and the controller may measure ICP to detect a blockage condition in the second catheter.

In accordance with another embodiment, an implant includes a pressure sensing assembly configured to communicate ICP and a valve assembly configured to block or pass the flow of CSF from a first catheter to a second catheter connected to the valve assembly. The pressure sensing assembly may include a body defining an upper surface and a lower surface. The lower surface is configured to rest on an outer surface of a skull. In an embodiment, the body is dimensioned to be seated over the burr hole and extend past an outer periphery thereof. In an embodiment, a conduit or rod extends from the lower surface of the body and into a subarachnoid space within the cranium or into a brain's parenchyma via the burr hole. The rod is configured to fluidly communicate ICP.

In accordance with another embodiment, a system for treating hydrocephalus includes a first catheter, a second catheter, and an implant including a controller, a pressure sensor, a tilt sensor, and a valve assembly operatively coupled to the first and second catheters. The first catheter may include a first lumen and a second lumen. The first lumen may define a fluid channel to drain or convey CSF away from the brain's ventricles, and the second lumen may define a fluid column to communicate pressure to the pressure sensor. The controller includes logic to determine ICP based on the pressure communicated via the fluid column. In an embodiment, the controller may include logic to determine whether the first catheter is disconnected based on the pressure communicated via the fluid column. In an embodiment, controller may receive the pressure communicated via the fluid column and a tilt sensor reading to determine the ICP to account for posture changes or patient positioning.

In accordance with an embodiment, a system for treating hydrocephalus may include an implant including a first catheter, a second catheter, and a valve assembly operatively coupled with the first and second catheters. In an embodiment, the implant includes a controller, a power supply, and a shape memory alloy (SMA) valve. The SMA valve may include a piston or plunger, and a first actuator and a second actuator operatively connected to the plunger. The first actuator and the second actuator may be configured to respectively open and close an inlet of the SMA valve by moving or pulling the plunger between an open position and a closed position. In an embodiment, the power supply may apply current to the first actuator to retract the first actuator, thereby causing the first actuator to move the plunger toward an open position. In an embodiment, the power supply may apply current to the second actuator to retract the second actuator, thereby causing the second actuator to move the plunger toward the closed position. In an embodiment, the SMA valve may include a first magnet and a second magnet respectively associated with the open and closed positions thereof. In an embodiment, the first magnet is configured to magnetically attract the plunger to maintain the valve assembly in the open position, and the second magnet is configured to magnetically attract the plunger to maintain the valve assembly in the closed position. In an embodiment, the controller can determine whether the SMA valve is in the open or closed position based on electrical resistance readings provided by the first and second actuators.

In accordance with an embodiment, a system for treating hydrocephalus in a patient includes a valve assembly and an external device. The valve assembly includes controller that actuates the valve assembly between an open position and a closed position, where the valve assembly conveys cerebrospinal fluid (CSF) from an intracranial space of the patient in the open position. The valve assembly also includes one or more sensors operatively connected to the controller, where the one or more sensors generate internal sensor data indicating a condition of the patient, and the controller actuates the valve assembly based on the internal sensor data. The valve assembly also includes a battery operatively connected to the one or more sensors or the controller. The external device is in wireless communication with the valve assembly. The external device generates an audible or visual warning, alarm, or notification when an amount of charge in the battery is below a predetermined threshold, the controller, the external device, or a remote server determine an event occurrence based on the internal sensor data, a manual override of the valve assembly exceeds a predetermined duration, or the external device attempts and fails to communicate with the valve assembly.

In accordance with an embodiment, a method for treating hydrocephalus in a patient, including conveying cerebrospinal fluid (CSF) from an intracranial space of the patient to a valve assembly through a proximal catheter. The method also includes generating internal sensor data with one or more sensors in the valve assembly, where the internal sensor data indicates an intracranial pressure (ICP) of the patient. The method also includes transmitting atmospheric pressure data from an external device or a remote server to the valve assembly, and transmitting operational data from the valve assembly to the external device. The method also includes deriving a gauge ICP based on the internal sensor data and the atmospheric pressure data collected at the valve assembly. The method also includes actuating the valve assembly between an open position and a closed position based on the gauge ICP, or generating an audible or visual warning, alarm, or notification with the external device when the external device attempts and fails to wirelessly communicate the atmospheric pressure data to the valve assembly a predetermined number of times or over a predetermined duration, or generating the warning, alarm, or notification when an amount of charge in a battery operatively connected to the valve assembly or the external device reduces below a predetermined threshold.

In accordance with an embodiment, a system for treating hydrocephalus in a patient includes a valve assembly and an external device. The valve assembly includes a controller that actuates the valve assembly between an open position and a closed position, where the valve assembly conveys cerebrospinal fluid (CSF) from an intracranial space of the patient in the open position. The valve assembly also includes one or more sensors operatively connected to the controller, the one or more sensors including a first pressure sensor that generates internal sensor data indicating an absolute intracranial pressure (ICP) of the patient, where the controller actuates the valve assembly based on the internal sensor data. The valve assembly also includes a first battery operatively connected to the one or more sensors or the controller. The external device is in wireless communication with the valve assembly. The external device includes a second pressure sensor that generates atmospheric pressure data, and includes a second battery operatively connected to the second pressure sensor, where the external device transmits the atmospheric pressure data to the valve assembly, and the valve assembly transmits operational data to the external device. The controller derives a gauge ICP of the patient from the internal sensor data and the atmospheric pressure data, the controller actuates the valve assembly based on the gauge ICP, and the external device generates a warning, alarm, or notification based on the operational data, or based on a failed attempt to perform wireless communication with the valve assembly.

### DESCRIPTION OF THE DRAWINGS

The present teachings may be better understood by reference to the following detailed description taken in connection with the following illustrations, wherein:

FIG. 1 is a block diagram of an example system for treating hydrocephalus according to an embodiment.

FIG. 2 is a perspective view of an example implant according to an embodiment.

FIG. 3 illustrates various examples of external devices according to embodiments.

FIG. 4 is a block diagram of an example system for treating hydrocephalus according to another embodiment.

FIG. 5A illustrates various examples of external devices according to embodiments.

FIG. 5B illustrates an example user interface of an external device according to an embodiment.

FIG. 6 is a block diagram of external devices, an implant, and a host device communicatively coupled via a wireless communications network.

FIG. 7 is a block diagram of an example system for treating hydrocephalus according to another embodiment.

FIG. 8 is an example user interface of an external device according to an embodiment.

FIG. 9 is a block diagram of an example system for treating hydrocephalus according to another embodiment.

FIG. 10 is a block diagram of an example system for treating hydrocephalus according to another embodiment.

FIG. 11A is a perspective view of an example implant according to another embodiment.

FIG. 11B is a sectional view of a proximal catheter taken along line 11B-11B in FIG. 11A.

FIG. 12 is a flow chart of an example methodology for detecting blockage.

FIG. 13 is a flow chart of an example methodology for determining whether a catheter is connected to the implant according to an embodiment.

FIG. 14 is a block diagram of an example system for treating hydrocephalus according to another embodiment.

FIG. 15A is a sectional view of a valve assembly according to an embodiment.

FIG. 15B is a schematic view of a valve assembly according to another embodiment.

FIG. 16 is a flow chart of an example methodology for determining a valve assembly position according to an embodiment.

FIG. 17 is a flow chart of an example methodology for determining whether a valve assembly is open or closed according to an embodiment.

FIG. 18 is a block diagram of an example system for treating hydrocephalus according to another embodiment.

FIG. 19 is a perspective view of an example implant operatively connected to an example pressure sensing assembly according to an embodiment.

FIG. 20 is a perspective view of the pressure sensing assembly of FIG. 19.

FIG. 21 is a perspective view of an example valve assembly with a capsule according to an embodiment.

FIG. 22 is an illustration of a simulated ICP drop.

FIGS. 23-25 depict example methodologies according to various embodiments.

FIG. 26 is a diagram of an example system for treating hydrocephalus according to another embodiment.

FIG. 27 is a diagram of an example system for treating hydrocephalus according to another embodiment.

FIG. 28 is a diagram of an example system for treating hydrocephalus according to another embodiment.

The invention may be embodied in several forms without departing from its spirit or essential characteristics.

### DETAILED DESCRIPTION

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

Reference will now be made in detail to embodiments of the present teachings, examples of which are illustrated in the accompanying drawings. It is to be understood that other embodiments may be utilized, and structural and functional changes may be made without departing from the scope of the present teachings. Moreover, features of the embodiments may be combined, switched, or altered without departing from the scope of the present teachings, e.g., features of each disclosed embodiment may be combined, switched, or replaced with features of the other disclosed embodiments. In this disclosure, numerous specific details provide a thorough understanding of the subject disclosure. It should be understood that aspects of this disclosure may be practiced with other embodiments not necessarily including all aspects described herein, etc. As such, the following description is presented by way of illustration and does not limit the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggest otherwise.

Throughout this disclosure ‘proximal’ means towards the brain and ‘distal’ away from the brain. In an embodiment, a proximal catheter drains CSF from the ventricle and a distal catheter empties the CSF into an abdominal peritoneum of a patient. However, other configurations, such as drainage from the subarachnoid space, or discharge into the right atrium or pleural sac, are also possible based on the present teachings. The present disclosure is not limited to the location of discharge. What is described herein is exemplary and any appropriate location of discharge within or even outside of the body may be utilized without departing from the present teachings.

“Logic,” synonymous with “circuit” as used herein, includes but is not limited to hardware, firmware, software and/or combinations of each to perform a function(s) or an action(s). For example, based on a desired application or needs, logic may include a software controlled microprocessor, discrete logic such as an application specific integrated circuit (ASIC), or other programmed logic device and/or controller. Logic may also be fully embodied as software.

“Software”, as used herein, includes but is not limited to one or more computer readable and/or executable instructions that cause a computer, logic, or other electronic device to perform functions, actions, and/or behave in a desired manner. The instructions may be embodied in various forms such as routines, algorithms, modules or programs including separate applications or code from dynamically linked libraries. Software may also be implemented in various forms such as a stand-alone program, a function call, a servlet, an app, instructions stored in a memory, part of an operating system or other type of executable instructions. It will be appreciated by one of ordinary skill in the art that the form of software is dependent on, for example, requirements of a desired application, the environment it runs on, and/or the desires of a designer/programmer or the like.

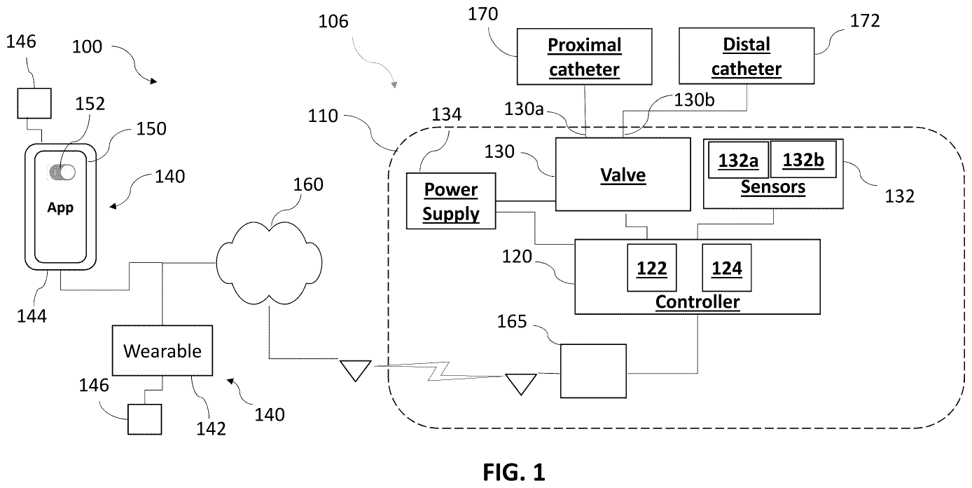

Referring to FIG. 1, an example system 100 for treating hydrocephalus comprises an implant 106 with a valve assembly 110, a proximal catheter 170, a distal catheter 172, and one or more external devices 140 communicatively coupled to the implant 106 via a communications network 160.

To facilitate communication, the valve assembly 110 may include a communications device 165 for sending and receiving data from the external devices 140. In some examples, the communications device 165 may include one or more controllers for standards-based networks (e.g., 2G, 3G, 6G, 6G LTE, 5G, GSM, UMTS, LTE, CDMA, WiMAX, etc.), satellite communication networks, and/or wireless local area networks (e.g., WiFi®, Wireless Gigabit, etc.), etc. In some examples, the communication device 165 may include controllers for personal area networks (e.g., ZigBee® (“IEEE 802.15.4”), Near Field Communication (“NFC”), etc.) to communicatively couple the valve assembly 110 to the external devices 140. In some embodiments, the communications device 165 may include a Bluetooth device (e.g., antenna and transceiver), or facilitate Wi-Fi-based communication such as via frequencies defined by the IEEE 802.11 standards, short-range wireless frequencies such as Bluetooth®, or any suitable wired or wireless communications protocol that facilitates communication between the valve assembly 110 and the external devices 140. In some embodiments, the communications device 165 may include a RFID antenna to wirelessly communicate with the external devices 140. It is also contemplated that the components of the valve assembly 110 may wirelessly communicate with each other via RFID technology.

The valve assembly 110 may include a controller 120, a valve 130, and one or more sensors 132 operatively connected to the controller 120. The controller 120 may embody a microcontroller that can send or receive data from the one or more sensors 132 to control an operation of the valve 130. The controller 120 may include a processor 122 and a storage device 124. The processor 122 may embody any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The storage device 124 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, non-volatile solid-state memory, etc.), unalterable memory (e.g., EPROMs), read-only memory, etc.). In some examples, the storage device 124 includes multiple kinds of memory, particularly volatile memory and non-volatile memory. The storage device 124 may also embody a computer readable medium on which one or more sets of instructions are embedded. The instructions may embody one or more of the methods or logic as described herein. In a particular embodiment, the instructions may reside completely, or at least partially, within any one or more of the storage device 124, the computer readable medium, and/or within the processor 122 during execution of the instructions.

The sensors 132 may include environment sensors (e.g., temperature sensors, humidity sensors, pressure sensors, accelerometers, light sensors, etc.). In an embodiment, the sensors 132 may include a pressure sensor 132a configured to provide ICP (e.g., absolute ICP) as a condition of the patient in real time. The sensors 132 generate internal sensor data, such as absolute intracranial pressure (ICP) and orientation data (e.g., tilt angle), which is used by the controller 120 to actuate the valve assembly.

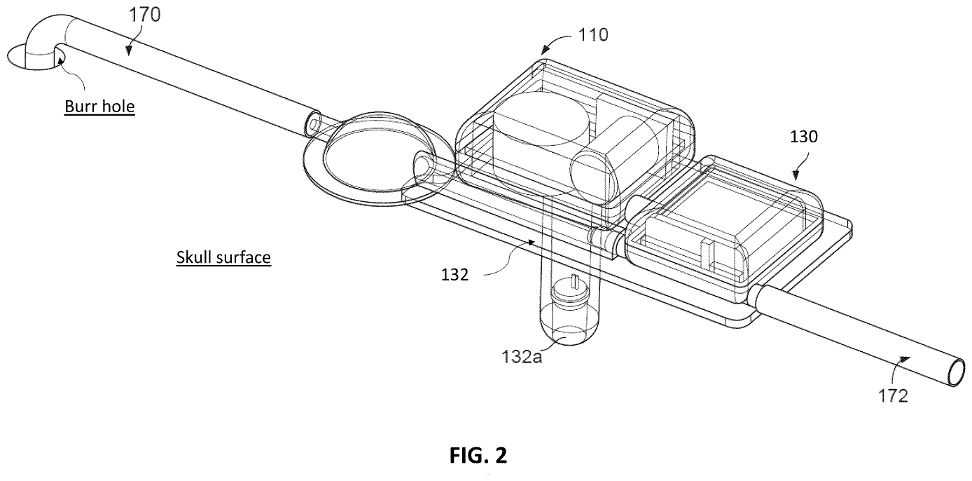

For example, referring to FIG. 2, the pressure sensor 132a may be disposed in the end of a rigid tube extending below the implant to measure ICP in the subarachnoid space just below the dura, or somewhat deeper, inside the brain's parenchyma. It is also contemplated that pressure sensor 132 may be disposed in a capsule tethered to the implant, or be disposed at an end of a lumen, for example, in a catheter with a dual lumen structure, i.e., a first lumen for conveying/draining CSF, and a second lumen for communicating pressure to the pressure sensor via a fluid column. In these embodiments, the pressure sensor 132a may be configured to measure and provide ICP at a location outside of a fluid channel for conveying/draining CSF. Separating the ICP measurement point from the fluid channel may facilitate detection of potential obstruction conditions of the fluid channel.

In some embodiments, the sensors 132 may also include a tilt sensor 132b configured to provide an angle of orientation relative to gravity as a real time condition of the patient. In some embodiments, the pressure sensor 132a may include a low-drift, media compatible pressure sensor configured to measure absolute ICP, and the tilt sensor 132b may include a 3-axis chip accelerometer configured to measure the implant patient's posture relative to gravity. The low-drift media compatible pressure sensor 132 may also be encapsulated in a high durability and biocompatible material such as Titanium.

In an embodiment, the controller 120 applies a pressure offset to the internal sensor data indicating absolute ICP based on preset calibration data associated with an orientation of the patient relative to gravity, as determined by the tilt sensor 132b. The controller 120 references the calibration data to correct the ICP measurement for posture-dependent hydrostatic effects, and then derives the gauge ICP by further accounting for ambient atmospheric pressure from an external device, such as the wearble device 142 or the mobile device 144. The calibration data may be determined noninvasively in a clinical setting by recording ICP readings while the patient's head is positioned in various orientations to gravity, including upright, supine, prone, and lateral decubitus. With this construction, the controller 120 enables accurate, posture-compensated ICP measurement and precise regulation of cerebrospinal fluid (CSF) drainage.

The valve 130 may include an inlet 130a operatively coupled to a proximal catheter 170 (e.g., a first catheter), and an outlet 130b operatively coupled to a distal catheter 182 (e.g., a second catheter). The proximal catheter 170 may be configured to convey cerebrospinal fluid (CSF) to the inlet 130a, and the distal catheter 172 may be configured to convey the CSF to another portion of the patient's body outside the intracranial space, e.g., into the abdominal peritoneum thereof. The valve 130 may be configured to reside on top of the implanted patient's skull, for example, under the scalp. Operatively, the valve 130 is configured to allow or block the flow of CSF from the inlet 130a to the outlet 130b based on instructions from the controller 120, e.g., to open or close the valve assembly based on ICP data. In some embodiments, the valve 130 may include an electromechanical latching valve based on shape memory alloy technology to provide silent switching, small size, and low power. In some embodiments, the sensors 132, power supply 134 and controller 120 may form part of the valve 130. It is also contemplated that each of the sensors 132, valve 130, and controller 120 may embody separate elements or form part of one or more subassemblies.

Referring to FIG. 1, a power supply 134 may be operatively connected to each of the valve 130, the sensors 132, the controller 120, and the communications device 165 to supply power thereto. The power supply 134 may embody a battery (e.g., a rechargeable high-density lithium cell). Wireless charging and power management circuitry may be included in the valve assembly 110 to ensure a safe, reliable recharge from an external charger device.

In some embodiments, the system 100 is configured to monitor an amount of charge remaining in the power supply 134. In this regard, when the charge drops below a predetermined threshold, the system 100 may generate a warning, alarm, or notification to alert the patient or caregiver. This warning may be generated by the external device 140, such as a wearable or mobile device, and may take the form of an audible sound, a visible light, or both. The intensity or modality of the warning generated at the external device 140 may escalate as the charge in the power source 134 continues to decrease, for example, starting with a low-intensity sound and progressing to a higher-intensity sound or a flashing light if the power source 134 is not recharged. This feature ensures that users including the patient or clinician are promptly notified of low battery charge conditions at the power supply 134, allowing timely recharging or replacement to maintain continuous operation of the valve assembly 110. In an embodiment, the external device 140 initially generates the warning, alarm, or notification including an audible sound having a first intensity, then generates the warning, alarm, or notification including an audible sound having a second intensity greater than the first intensity after a first predetermined period of time, and then generates the warning, alarm, or notification including a visible light after a second predetermined period of time following the first predetermined period of time.

In various embodiments, the system 100 includes the power supply 134 as a first battery operatively connected to the valve assembly 110, and includes a second battery operatively connected to the external device 140. The system 100 is configured to independently monitor the charge level of both the first battery and the second battery, and generate warnings or alarms in response to the charge level of either the first battery or the second battery falling below predetermined thresholds. In this regard, when the charge level of either the first battery in the valve assembly 110 or the second battery in the external device 140 falls below progressively lower predetermined thresholds, the external device 140 generates a warning, alarm, or notification with increasing intensity and various modalities as described above. In an embodiment, the external device 140 generates the warning, alarm, or notification with increased intensities and various modalities as described above, after successive predetermined periods of time have elapsed. The external device 140 may generate the warning, alarm, or notification repeatedly or continuously in response to detecting that the amount of charge in the first battery or the second battery is below any of the predetermined thresholds, and discontinues generating the warning, alarm, or notification when the external device detects the first battery or the second battery is charging or has been recharged.

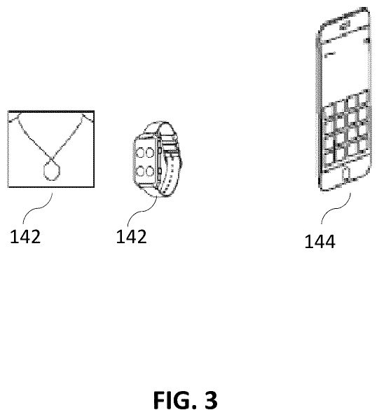

As noted above, one or more external devices 140 may be communicatively coupled with the valve assembly 110. Referring to FIG. 3, the external device 140 may embody, for example, a smartphone 144, a tablet, or a notebook, or a wearable device 142 worn by the user (e.g., a smartwatch or other suitable device including a processor, a sensor, and a transceiver (e.g., a BLE transceiver circuit) for receiving or sending data to the valve assembly 110. For example, the wearable device 142 may embody a necklace, a bracelet, a clip, a ring, a pin, a clip, a belt buckle (or attachment therefor), or a puck-shaped device (e.g., that may be stored in the user's pocket), each including a processor, a sensor, and a transceiver. In some embodiments, the external device 140 may embody a host device (e.g., a clinic device) that is a remote terminal hosting a clinic app that is used by clinicians (certified medical specialists e.g. doctors and nurses). The clinic app may generally communicate with the wearable, with the implant, or with a cloud server to exchange data with the other devices. The clinic app may act as a portal to the cloud database. It may allow clinicians to access a given patient's historical data. It may filter said data using thresholds and parameters set by the clinician, or default thresholds and parameters. It may graphically display the data and perform statistical analysis of it, for example averages, standard deviations, variance analysis. It may identify longitudinal trends in the patient's data, for example long-term changes in daily ICP over time. It may include search capability allowing the clinician to use search criteria to filter, correlate and display data—for example, a clinician may want to find average ICP during nighttime versus daytime, or may wish to see the patient's average “valve open” time per day per month and time to restore normal ICP for each ‘valve open’ event for the past 3 years. The clinical app may allow correlation of data with external events that are either reported by the patient, patient caregivers, clinicians, the patient's electronic medical record, or the Smartshunt system itself. External events may include any of the ‘event data’ parameters listed elsewhere in this application, and may further include data from other sources such as the patient's medical record. The clinical app may contain learning algorithms or artificial intelligence algorithms that may predict future physiological outcomes based on past outcomes, and may recommend or implement systems settings changes based on these. It may allow the physician to change device settings remotely, for example valve open ICP threshold, valve close ICP threshold, time of ICP averaging window, valve ‘safe state’ in case of power loss (open or closed). The clinic app may be configured to recommend settings and treatment based on accepted medical guidelines and on the individual patient's physiological status and measurements. The clinic app may alert clinicians to Smartshunt system health conditions that may require attention, for example suspected shunt obstruction, low battery, battery end-of-lifetime, suspected system malfunction, inability to contact wearable, internal system faults, etc. The clinic app may connect to a larger database of SmartShunt patients who under the care of other clinicians. The larger database may anonymize patient data to comply with Protected Health Information standards such as HIPAA and the EU General Data Protection Regulation. The clinic app may use data from the larger database to allow clinicians to see how their patients compare to the larger hydrocephalus population, as well as how other physicians are using the system (adjusting settings, etc) and their outcomes. The clinic app may provide similar search and correlation functions to the larger database as to the database from the clinicians' own patients.

In some embodiments, the system 100 includes a remote server operatively coupled to the external devices 140, and a remote terminal operatively coupled to the remote server as one of the external devices 140. The external device 140 including the wearable device 142 or the mobile device 144 transmits internal sensor data, external sensor data, or operational data from the valve assembly 110 or the external device 140 to the remote server repeatedly within a predetermined interval, such as every 5 minutes, every hour, or at another interval set by the user or clinician. The predetermined interval may be adjustable based on clinical requirements or user preferences. The remote terminal may include a computer, tablet, smartphone, or other device operated by a clinician, caregiver, or system administrator, and is configured to monitor the remote server for updates or new data received from the wearable device 142 or the mobile device 144. The remote terminal may periodically query or receive notifications from the remote server to determine whether the wearable device 142 or the mobile device 144 has communicated the expected data within the predetermined interval.

When the remote terminal determines that the wearable device 142 or the mobile device 144 has not communicated the internal sensor data, the external sensor data, or the operational data to the remote server within the predetermined interval, the remote terminal generates a warning, alarm, or notification. This warning, alarm, or notification may be visual (e.g., a pop-up notification or flashing indicator on the remote terminal display), audible (e.g., a beep or alarm sound), or both, to alert the user or clinician to the communication failure between the valve assembly 110, the wearable device 142, and the mobile device 144. The warning, alarm, or notification may persist or escalate until the communication issue is resolved or acknowledged by the user. In some embodiments, the remote terminal may also display information regarding the time of the last successful data transmission, the type of data expected, and suggested troubleshooting steps.

In embodiments, the external device 140 includes a stationary hub located in the patient's home, a clinic, or another fixed environment where the patient regularly spends time. This hub continuously or periodically establishes wireless communication with the valve assembly 110 whenever the patient is within range, enabling the transfer of internal sensor data, operational data, and status updates from the implant to the hub. The stationary hub is equipped with a processor, memory, and a wireless transceiver (such as Bluetooth® or Wi-Fi®), and may be powered by a continuous power source or rechargeable battery. After receiving data from the valve assembly 110, the hub transmits the data to a remote server via a wired or wireless internet connection at predetermined intervals, which may be set according to clinical requirements or user preferences. In this manner, the hub serves as a local, stationary communication point, and ensures reliable and secure data transfer between the implantable valve assembly 110 and the remote server, even if the patient does not carry a mobile device at all times. Furthermore, the hub may supplement communications with the valve assembly 110 and the remote server or other external devices 140, including where a patient has limited access to mobile technology or where continuous mobile connectivity is not feasible. The fixed location of the hub maintains a stable connection to the remote server and provides a consistent data relay whenever the patient is present within its wireless communication range.

This configuration offers several advantages. By serving as a local, stationary communication point, the hub ensures reliable and secure data transfer between the implantable valve assembly 110 and the remote server, even if the patient does not carry a mobile device at all times. This is particularly beneficial for patients with limited access to mobile technology or in settings where continuous mobile connectivity is not feasible. The hub's fixed location allows it to maintain a stable connection to the remote server and act as a consistent data relay whenever the patient is present within its wireless communication range.

Referring to FIG. 1, one or more atmospheric sensors 146 may be coupled to or embedded within each external device 140 to measure local atmospheric conditions, e.g., atmospheric pressure, temperature, and the like. For this purpose, each external device 140 may include a barometer or a temperature sensor (e.g., that measures the battery or CPU temperatures of the external device). As such, the external devices 140 may be equipped with sensors such as barometers and temperature sensors that generate external sensor data associated with the patient, including ambient atmospheric pressure and temperature, which can be transmitted to the valve assembly 110 for use in determining gauge ICP and other operational parameters.

A software application (“app”) 150 may reside on the external device 140, for example, on the patient's or another person's smartphone 144. The clinical app 150 may include a computer program or software including a set of instructions. The instructions may embody one or more of the methods or logic as described herein. The clinical app 150 may be configured to receive or send data to the valve assembly 110 (e.g., to the controller 120 thereof) via the external device 140. In an embodiment, the data may include atmospheric data, for example, an ambient pressure value or temperature, and the like. In such embodiments, it is contemplated that the clinical app 150 may receive the data via a barometer or temperature sensor residing in the external device 140. It is also contemplated that the clinical app 150 may be communicatively coupled to another app, a website, or an external server to receive atmospheric data. In some embodiments, the clinical app 150 is publicly available so that anyone may download it on to their respective external device 140 for use with the valve assembly 110.

In some embodiments, an implant user may have more than one wearable device 142 configured to provide atmospheric data to the valve assembly 110, for example, a first wearable device 142 to communicate with the valve assembly 110 while a second wearable device 142 is charging.

In some embodiments, the controller 120 (e.g., firmware thereof) may include logic to listen for and communicate with the wearable device 142, for example, to receive atmospheric data therefrom every 3 to about 12 seconds, or about every 8 seconds, for example. In this manner, the controller 120 may include logic to determine a gauge ICP (e.g., gauge average ICP value) of the patient based on the atmospheric pressure (obtained via the external device 140) and an absolute ICP value (e.g., absolute average) received from the sensors 132. The controller 120 may determine the gauge ICP based on additional data, for example, tilt angle data (provided by the tilt sensor 132b) to account for fluid column weight offsets based on the patient's posture. In an embodiment, the implant may stay in a low-power sleep mode for a present interval, and wake itself up using an internal timer circuit. It may then listen for the ambient pressure data from the Wearable as described above.

It is contemplated that communication between a user's wearable device 142 and the valve assembly 110 may be lost, for example, due to a communication network outage, or because the wearable device 142 is not charged or has been lost or damaged by the patient. In such embodiments, the controller 120 may listen for the external device 140, but not receive a reading (e.g., atmospheric data) therefrom. For this purpose, the user may utilize the clinical app 150 on another external device 140 (e.g., a smartphone 144) to provide atmospheric data to the valve assembly 110 (e.g., to the controller 120 thereof). In this manner, the clinical app 150 (downloaded on the smartphone 144) may serve as a backup to provide atmospheric data to the controller 120 when the controller 120 is unable to receive atmospheric data from the user's wearable device 142. In this manner, the clinical app 150 may provide atmospheric data to the controller 120 after it has failed to receive it from the wearable device 140 within a predetermined duration such as, for example, at about 30 to 90 seconds, or about 60 seconds after failing to communicate with the wearable device 142. In some embodiments, the controller 120 may communicate with the clinical app 150 to receive atmospheric data therefrom after a certain number of attempts to reach the user's wearable device 142, for example, after 10 or 100 attempts or more. In some embodiments, the system 100 may be setup such that the clinical app 150 is configured to transmit atmospheric data to the valve assembly 110 at an interval that is less than the implant's search time, i.e., the time to search for the wearable device 142. For instance, the valve assembly 110 may performed periodic wake cycles where the valve assembly 110 searches for the wearable device 142 to receive atmospheric data therefrom every 10 seconds, while the clinical app 150 is configured to transmit atmospheric data to the implant every second. With this construction, the wake cycles by the valve assembly 110 occur at an interval shorter than an interval at which the mobile device 144 periodically transmits the external sensor data, optionally in a beacon mode. Having the wake cycles of the valve assembly 110 occur at an interval shorter than the interval at which the mobile device 144 periodically transmits external sensor data increases a likelihood that the valve assembly 110 will be awake and able to receive the transmitted data, thereby ensuring reliable and timely receipt of critical sensor information for system operation while also conserving an overall charge in the power supply 134.

In some embodiments, the clinical app 150 may feature a user interface or input device 152 to enable the clinical app 150 to send atmospheric data to the valve assembly 110, for example, a slider, a toggle button, a voice activated command, etc. In some embodiments, the clinical app 150 may be downloaded via any smartphone (e.g., Android or Apple® devices), thereby providing the user a variety of options to provide atmospheric data to the implant. In an embodiment, the display will simply state that the ambient pressure is currently being broadcast and may state the frequency of broadcasting. It may display the ambient pressure. In embodiments where the clinical app 150 carries out 2-way communications with the valve assembly 110, the display may include acknowledgements from the valve assembly 110 that the ambient pressure data was received.

In some embodiments, the patient's wearable device 142 or other external device 140 (e.g. smartphone 144) may alert the user that their wearable device 142 has not successfully transmitted ambient data to the valve assembly 110 within a preset time limit, for example, via an audible or visible warning, alarm, or notification. This may prompt the patient to ask a family member, friend, or another person (e.g., a person on a plane or bus, a person at work or school etc.) to download the clinical app 150 onto that person's external device 140 (e.g. smartphone 144) to transmit atmospheric data to the user's valve assembly 110 as a temporary backup option, until the patient finds their wearable device 142, or until it is again communicatively coupled with the valve assembly 110. In further embodiments, if the valve assembly 110 fails to receive external sensor data from the wearable device 142 within a predetermined period, the system 100 may prompt the patient, for example via an audible, visual, or haptic alert on the wearable device 142, the mobile device 144, or a second wearable device, to simultaneously activate both the wearable device 142 and the mobile device 144 or second wearable device. The prompt may instruct the patient to ensure that both devices are powered on and within wireless communication range of the valve assembly 110 and each other. This coordinated activation facilitates re-establishing robust communication links and enables the system to synchronize data transmission and reception among each of the valve assembly 110, the wearable device 142, and the mobile device 144. If, after a further predetermined period, the mobile device 144 does not detect successful activation and communication from both devices, the mobile device 144 may generate additional reminders or escalation alerts may be issued to the patient or caregiver. The mobile device 144 may additionally or alternatively prompt the patient to periodically connect the valve assembly 110, the wearable device 142, and the mobile device 144, such as once a day.

The valve assembly 110 may transmit internal sensor data or first local operating data to both the wearable device 142 and the mobile device 144 or second wearable device in response to detecting the activation. The wearable device 142 and the mobile device 144 or second wearable device may then transmit external sensor data or second local operating data to each other and the valve assembly 110, ensuring synchronized data exchange and robust communication in the system 100.

This aspect of the present disclosure may be beneficial for situations, where the implant patient is expected to experience pressure changes, for example, a pressure reading before a plane descends and then a subsequent reading while on the plane at a higher altitude. Members of a patient's family, friends, caregivers, school, workplace, may be instructed to have the clinical app 150 available in case the patient requires it due to a lost or damaged wearable. Entities that routinely expose the public to significant ambient pressure changes, such as airlines, reception staff at tall buildings, amusement parks, etc., may also be instructed to have the clinical app 150 available.

In some embodiments, the clinical app 150 may reside on the user's wearable device 142 and the clinical app 150 may be used for each external device 140 (e.g., their wearable device 142 or their smartphone 144). In some embodiments, the clinical app 150 may be unique to the patient's valve assembly 110, for example, by only enabling communication between the clinical app 150 and the valve assembly 110 based on authentication, e.g., the user's credentials, serial number, etc. It is also contemplated that the clinical app 150 may include a unique, embedded token specific to the implant patient. The clinical app 150 may be configured to be highly simplified such that no pairing is needed. In this configuration the device 142 (e.g. smartphone 144) may operate in the Bluetooth “beacon mode”, in which the clinical app 150 simply broadcasts the ambient pressure periodically to any devices that are in range and listening for it.

In addition to transmitting atmospheric data to the valve assembly 110, the clinical app 150 may be operable to log data (e.g., symptoms the user is experiencing) that is stored in memory, e.g., any suitable example of a storage device or memory disclosed herein.

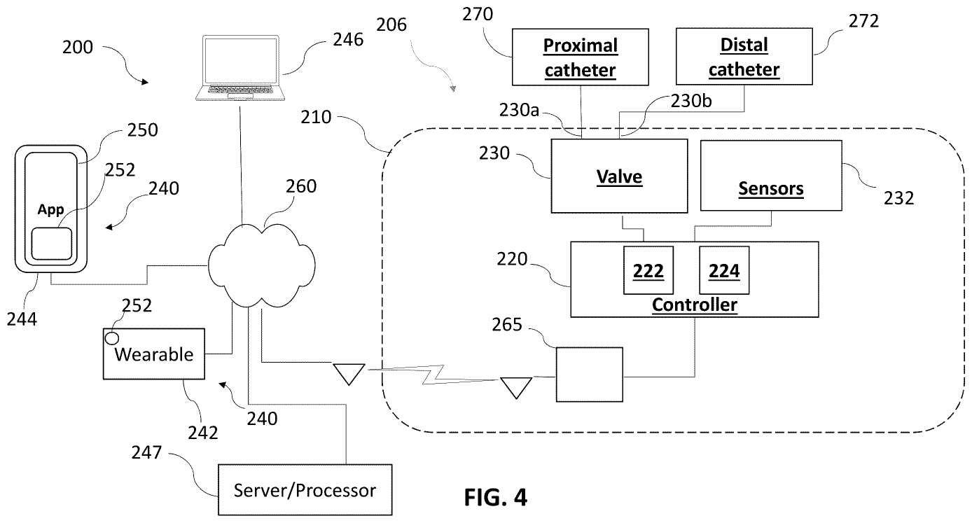

Referring now to FIG. 4, another example system 200 for treating hydrocephalus is shown. The system 200 includes an implant 206 with a valve assembly 210, a proximal catheter 270, and a distal catheter 272, and one or more external devices 240 communicatively coupled to the valve assembly 210 via a communications network 260.

To facilitate communication, the valve assembly 210 may include a communications device 265 for sending and receiving data from the external devices 240. In some examples, the communications device 265 may include one or more controllers for standards-based networks (e.g., 2G, 3G, 6G, 6G LTE, 5G, GSM, UMTS, LTE, CDMA, WiMAX, etc.), satellite communication networks, and/or wireless local area networks (e.g., WiFi®, Wireless Gigabit, etc.), etc. In some examples, the communication device 265 may include controllers for personal area networks (e.g., ZigBee® (“IEEE 802.15.4”), Near Field Communication (“NFC”), etc.) to communicatively couple the valve assembly 210 to the external device 240. In some embodiments, the communications device 265 may include a Bluetooth device (e.g., antenna or transceiver), or facilitate Wi-Fi-based communication such as via frequencies defined by the IEEE 802.11 standards, short-range wireless frequencies such as Bluetooth®, or any suitable wired or wireless communications protocol that facilitates communication between the valve assembly 210 and the external device 240. In some embodiments, the communications device 265 may include an antenna to wirelessly communicate with the external device 240. It is also contemplated that the components of the valve assembly 210 may wirelessly communicate with each other.

The valve assembly 210 may include a controller 220, a valve 230, and one or more sensors 232 (e.g., a pressure sensor, tilt angle sensor, etc.) operatively connected to the controller 220. The controller 220 may embody a microcontroller that can send or receive data from the one or more sensors 232 to control an operation of the valve 230. The controller 220 may include a processor 222 and a storage device 224. The processor 222 may embody any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The storage device 224 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, non-volatile solid-state memory, etc.), unalterable memory (e.g., EPROMs), read-only memory, etc.). In some examples, the storage device 224 includes multiple kinds of memory, particularly volatile memory and non-volatile memory. The storage device 224 may also embody a computer readable media on which one or more sets of instructions are embedded. The instructions may embody one or more of the methods or logic as described herein. In a particular embodiment, the instructions may reside completely, or at least partially, within any one or more of the storage device 224, the computer readable medium, and/or within the processor 222 during execution of the instructions.

The valve 230 may include an inlet 230a operatively coupled to a proximal catheter 270 (e.g., a first catheter), and an outlet 230b operatively coupled to a distal catheter 282 (e.g., a second catheter). The proximal catheter 270 may be configured to convey cerebrospinal fluid (CSF) to the inlet 230a, and the distal catheter 272 may be configured to convey the CSF to another portion of the implant patient's body, e.g., into the abdominal peritoneum thereof. Operatively, the valve 230 is configured to allow or block the flow of CSF from the inlet 230a to the outlet 230b based on instructions from the controller 220, e.g., to open or close the valve assembly based on ICP data derived from the sensors 232, for example, based on a detected, absolute ICP, a tilt angle of the patient relative to gravity, and atmospheric pressure (e.g., retrieved via the sensors 232 or via atmospheric sensors in the external devices 240).

The external devices 240 may embody, for example, a smartphone 244, a tablet, or a notebook, or a wearable device 242 worn by the user (e.g., a smartwatch or other suitable device including a processor, a sensor, and a transceiver (e.g., a BLE transceiver circuit)) for receiving or sending data to the valve assembly 210. For example, the wearable device 242 may embody a necklace, a bracelet, a clip, a ring, a pin, a clip, a belt buckle (or attachment therefor), a wristwatch, a smartphone, or a coin-or puck-shaped device (e.g., that may be stored in the user's pocket), each including a processor, a sensor, and a transceiver.

A software application (“app”) 250 may reside on some or all of the external devices 240, for example, on the patient's smartphone 244 or wearable device 242. In some embodiments, at least one of the external devices 240 may include a user interface or input device 252 operable to log event data. As used herein, event data is intended to refer to data associated with an event that may affect, be affected by, or be related to the patient's hydrocephalus. For example event data may include data associated with events the patient suffers or incurs such as headaches, dizziness, light-headedness, nausea, weight, electrocardiogram data, posture, swimming or diving, exercise, exertion, eating, sleeping, bowel movements, urination, medication changes, tobacco/alcohol/controlled substance use, strokes or seizures, pupil dilation, menstrual cycle, travel/move to new location, fatigue level, work/school schedule, home/life events (death in family, divorce, etc.), childbirth, medical or dental procedures, accidents, psychological stress, blood pressure (arterial, venous, or pulmonary artery), glucose level, hydration (including water intake), ambient pressure, ambient temperature, neural dysfunction (e.g. speech, hearing, vision or motor skill abnormalities), psychological conditions (e.g. anxiety, mood swings, etc.), head or other injuries, dementia symptoms, other medical conditions, states, or symptoms, etc. In embodiments, the event data may be utilized to correlate the events to ICP or make predictions concerning patient outcomes based on the data, e.g., via machine learning. In embodiments, the event data may be used by the neuroscience industry as a whole to discover new correlations and/or make predictions regarding hydrocephalitics. For example, it may be discovered that opening the valve relieves headaches for a specific patient, or a subset of patients, or that a patient or subset of patients may exhibit lightheadedness, despite corrective measures in place, for example, an implant with a valve assembly including logic to prevent this.

In embodiments, the event data, ICP, atmospheric data, or other data (e.g., made available from the valve assembly or external device) (collectively referred to as “operating data”) may be sent to a remote server including a processor 247 communicatively coupled with an implant, an external device, host device, or app. The processor may embody any suitable processing device or set of processing devices disclosed herein.

The processor 247 may receive the operating data and include logic to make insights or predictions regarding patient outcomes or implant performance, e.g., for a specific patient, a subset of patients, an implant manufacturer, or for the neurosciences industry as a whole. In embodiments, the remote server may receive the operating data to train and build a predictive model (e.g., via unsupervised machine learning) for making predictions concerning patient outcomes or implant performance, e.g., via artificial intelligence. For example, the processor may make predictions (e.g., concerning battery life, CSF flow rates, potential catheter blockages, and the like) based on the operating data. In some embodiments, the processor may utilize the operating data to make predictions regarding implant performance (e.g., in the future, e.g., after 2, 3, 4, or 10 years, for example). The processor may also utilize the operating data may to make predictions regarding a specific patient's CSF flow rates or ICP in the future or after time has passed since the implant was first operational. For example, the processor make predictions that the CSF flow rate or ICP for a particular patient may change in the future, for example, based on operating data including biometric data or clinical profile data that indicates the patient's age, height, weight, a projected growth (e.g., a projected increase in height or weight during adolescence) or any other suitable information specific to the patient (e.g., the patient's race or ethnicity). In some embodiments, the operating data may be supplemented with patient data, for example, data from the patient's medical records (e.g., available CT scans).

In an embodiment, the input device may be communicatively coupled to a global positioning system (GPS) device configured to determine a location of the implant patient during the event. The external device may be combined with other devices, such as the wearable used for ambient pressure and data communications described in the reference applications. The external device 240 may be a dedicated device, or may be an app on a 3^{rd} party device such as a smartphone or tablet. As a dedicated device, the external device 240 may be a dedicated, single-purpose device operating on a locked operating system that suspends, constrains, or disables automatic updates. With this construction, the external device 240 ensures consistent and reliable communication with the valve assembly 210, minimizing a risk of interruptions or failures due to software updates or other external factors.

The external device 240 may include a user interface 252 such as pushbutton, touchscreen, voice input or gesture input. The user interface 252 may further include a means for the user to input additional information about the logged event, for example severity of symptoms (e.g., scale 1-10), type of food eaten, etc. The user interface 252 may include an automated interface to one or more custom or 3^{rd} party activity logging apps as are known in the art, for example food log, exercise log, or location tracking apps. In this configuration the user interface 252 would automatically retrieve timestamped data from the activity logging apps. The external device 240 may be configured to timestamp the user input and add it to a datalog created by the system. The correlation of logged events with data logged by the system (pressure measurements, valve state, posture, etc.) may be carried out by dedicated software located in one of the devices or in a remote processor/server. The correlation of logged events and measured parameters may be used as input to machine learning or artificial intelligence algorithms.

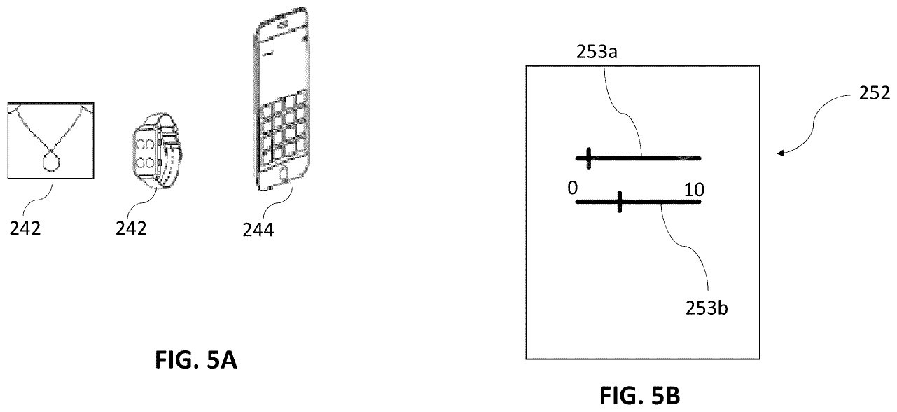

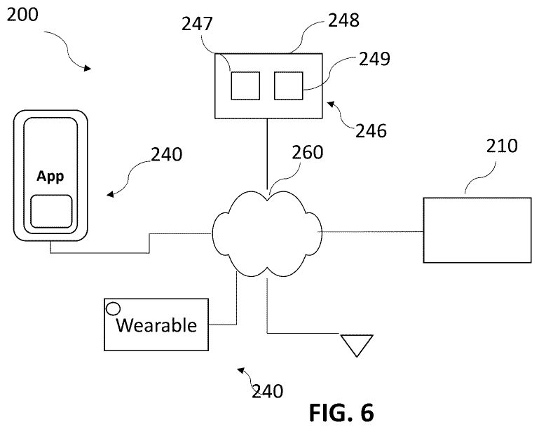

The input device 252 may form part of the external device 240 (e.g., a button or rotatable dial on the housing or enclosure of the external device), or a feature of a graphic user interface thereof. In addition or alternatively, the input device 252 may be accessible via the app 150 associated with the external device 240. Examples of an input device 252 may include, but are not limited to, an alphanumeric input device (e.g., a keyboard), a pointing device, a physical button or a touch screen button, an audio input device (e.g., a microphone, a voice response system, etc.), a cursor control device, a touchpad, a video capture device (e.g., a still camera, a video camera), a touchscreen, a text button, a dropdown button, a text box, a slider 253 (FIG. 5B), a toggle button, haptic or gesticulate inputs, and/or any combinations thereof. In some embodiments, it is contemplated that the input device 252 may be operable to define the severity rating associated with the event, for example, on a scale from 1-10 (FIG. 5B). In some embodiments, the input device 252 may include one or more inputs 253a and 253b for defining a severity rating for one or more symptoms, for example, a first input 253a for indicating the severity of a headache, and another input 253b for indicating the severity of lightheadedness.

The event data captured via the input device 252 may be supplemented with other event data, for example, the date and time of the medical event, the atmospheric conditions including temperature and atmospheric pressure (e.g., provided via sensors 232 or the external device 250), or the tilt angle, e.g., the tilt angle of the patient's head or thorax relative to gravity. In some embodiments, the event data may include or be supplemented by a voice recording of the user, for example, the implant patient speaking into a microphone on their external device 250 to describe the event or symptoms. In some embodiments, the external device 250 may transcribe the implant patient's voice recording into text, e.g., via a voice to text feature.

In some embodiments, the input device 252 may be communicatively coupled to a global positioning system (GPS) device configured to determine a location of the implant patient during the medical event. The patient's location may then be added to the event data.

The event data may be transmitted to other external devices or a host device 246 communicatively coupled with the external devices 240 via the communications network 260. The host device 246 may embody a smartphone, a table, a notebook, or any other suitable example of a external device described herein. The host device 246 may belong to a clinician who could review the event data associated with the medical event, in conjunction with implant data retrieved from the valve assembly 210, for example, the ICP, tilt angle, etc. Clinicians may include any licensed medical professional, such as doctors, nurses, nurse practitioners, physicians' assistants, etc. Specifically, neurosurgeons, neurologists and their staff members may include the Smartshunt's clinician user base.

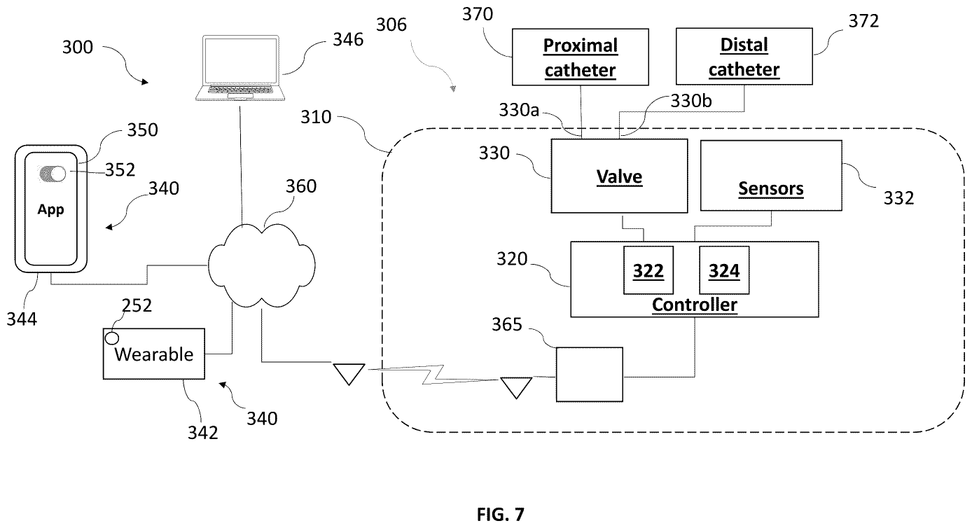

Referring to FIG. 6, in some embodiments, the host device 246 may include a controller 248 including a processor 247 and a storage device 249, for example, any suitable example of a processor and storage device described herein. In some embodiments, the controller 248 and storage device 249 may reside on a server communicatively coupled with the host device 246. The controller 248 may include logic to execute a set of instructions (e.g., residing in the storage device) to determine a relationship between the event data and the implant data. In some embodiments, the controller 248 may identify a correlation between ICP, CSF drainage events, patient recorded events (via the event data), and data from other sources such as other medical devices or the patient's electronic medical record. In some embodiments, the event data and the implant data may be used to train a machine learning model configured to predict a diagnosable medical event, for example, a severe headache attributed to an increase in ICP within the cranium. In some embodiments, the controller 220 (FIG. 4) in the valve assembly 210 may control the valve functionality based on the event data and the implant data, for example, to open or close the inlet 230a, or increase or decrease the flow rate of CSF flowing therethrough, e.g., by opening or closing the valve, or via a valve that throttles flow therethrough to adjust a flow rate of CSF. In some embodiments, the valve assembly 210 may control the valve based on operating data, for example, any suitable example described herein.

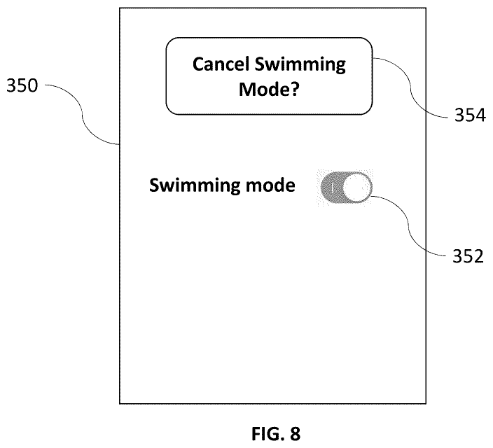

Referring now to FIG. 7, another example system 300 for treating hydrocephalus is shown. The system 300 includes an implant with a valve assembly 310, a proximal catheter 370, a distal catheter 372, and one or more external devices 340 communicatively coupled to the valve assembly 310 via a communications network 360.

To facilitate communication, the valve assembly 310 may include a communications device 365 for sending and receiving data from the external devices 340. In some examples, the communications device 365 may include one or more controllers for standards-based networks (e.g., 2G, 3G, 6G, 6G LTE, 5G, GSM, UMTS, LTE, CDMA, WiMAX, etc.), satellite communication networks, and/or wireless local area networks (e.g., WiFi®, Wireless Gigabit, etc.), etc. In some examples, the communication device 365 may include controllers for personal area networks (e.g., ZigBee® (“IEEE 802.15.4”), Near Field Communication (“NFC”), etc.) to communicatively couple the valve assembly 310 to the external device 340. In some embodiments, the communications device 365 may include a Bluetooth device (e.g., antenna or transceiver), or facilitate Wi-Fi-based communication such as via frequencies defined by the IEEE 802.11 standards, short-range wireless frequencies such as Bluetooth®, RFID, or any suitable wired or wireless communications protocol that facilitates communication between the valve assembly 310 and the external device 340. It is also contemplated that the components of the valve assembly 310 may wirelessly communicate with each other via RFID technology.

The valve assembly 310 may include a controller 320, a valve 330, and one or more sensors 332 (e.g., a pressure sensor, tilt angle sensor, etc.) operatively connected to the controller 320. The controller 320 may embody a microcontroller that can send or receive data from the one or more sensors 332 to control an operation of the valve 330. The controller 320 may include a processor 322 and a storage device 324. The processor 322 may embody any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The storage device 324 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, non-volatile solid-state memory, etc.), unalterable memory (e.g., EPROMs), read-only memory, etc.). In some examples, the storage device 324 includes multiple kinds of memory, particularly volatile memory and non-volatile memory. The storage device 324 may also embody a computer readable media on which one or more sets of instructions are embedded. The instructions may embody one or more of the methods or logic as described herein. In a particular embodiment, the instructions may reside completely, or at least partially, within any one or more of the storage device 324, the computer readable medium, and/or within the processor 322 during execution of the instructions.

The valve 330 may include an inlet 330a operatively coupled to a proximal catheter 370 (e.g., a first catheter), and an outlet 330b operatively coupled to a distal catheter 382 (e.g., a second catheter). The proximal catheter 370 may be configured to convey cerebrospinal fluid (CSF) to the inlet 330a, and the distal catheter 372 may be configured to convey the CSF to another portion of the implant patient's body, e.g., into the abdominal peritoneum thereof. Operatively, the valve 330 is configured to allow or block the flow of CSF from the inlet 330a to the outlet 330b based on instructions from the controller 320, e.g., to open or close the valve assembly based on ICP data derived from the sensors 332, for example, ICP data derived from an absolute ICP, a tilt angle of the patient (e.g., the patient's thorax or a catheter relative to gravity), or atmospheric pressure (e.g., retrieved via the sensors 332 or via the external devices 340).

The external device 340 may embody a smartphone 344, a tablet, a notebook, or a wearable device 342 worn by the user (e.g., a smartwatch or other suitable device including a processor, a sensor, and a transceiver (e.g., a BLE transceiver circuit)) for receiving or sending data to the valve assembly 310. For example, the wearable device 342 may embody a necklace, a bracelet, a clip, a ring, a pin, a clip, a belt buckle (or attachment therefor), or a puck-shaped device (e.g., that may be stored in the user's pocket), each including a processor, a sensor, and a transceiver. In some embodiments, the external device 340 may include a host device (e.g., a clinic device) hosting clinic app.

A software application (“app”) 350 may reside on some or all of the external devices 340, for example, on the user's smartphone 344 or wearable device 342. In some embodiments, at least one of the external devices 340 may include a user interface or input device 352. The input device 352 may form part of the external device 340 (e.g., a button or rotatable dial on the housing or enclosure of the external device), or a feature of a graphic user interface thereof. In addition or alternatively, the input device 352 may be accessible via the app 350 associated with the external device 340. Examples of an input device 352 may include, but are not limited to, an alphanumeric input device (e.g., a keyboard), a pointing device, a physical button or a touch screen button, an audio input device (e.g., a microphone, a voice response system, etc.), a cursor control device, a touchpad, a video capture device (e.g., a still camera, a video camera), a touchscreen, a text button, a dropdown button, a text box, a slider, a toggle button, and/or any combinations thereof. With this construction, the wearable device 342 may be smaller and more portable as compared to the smartphone 344 or other mobile device.

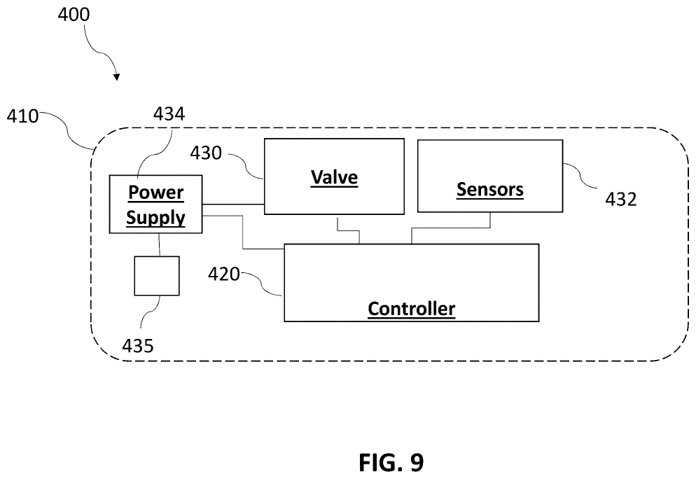

In some embodiments, it is contemplated that the input device 352 may be operable to actuate a disable or swimming mode (i.e., an override mode), to inform the controller 320 that the user will soon be exposed to significant, transient atmospheric pressure changes, for example, when diving into water, which adds about 74 mmHg of ambient pressure for every meter underwater. The controller 320 may receive this communication from the user via input device 352. At the next data communication between valve assembly 310 and external device 340 (for example the timed pressure reading and data exchange between valve assembly 310 and an external wearable device described herein), the external device 340 may send a “Swim Mode” signal to the valve assembly 310. The valve assembly 310 may react to the Swim Mode signal by altering its ‘timed reading’ algorithm until a “Cancel Swim Mode” signal is received from the user via external device 340. In one embodiment, the altered algorithm may simply suspend timed readings until the “Cancel Swim Mode” signal is received; in this embodiment it may place the valve in its designated safe state (open or closed as determined by physician setting, with default closed). In another embodiment, the altered algorithm may take the timed ICP reading as described elsewhere, but when computing the average ICP from samples taken throughout the measurement time window, it may ignore all samples that are greater than a certain threshold, for example 15 mmHg, above the ambient reading provided by the external device 340. The reading rejection threshold may be a value adjustable in the device settings by the physician. Typical values may be between 5 and 20 mmHg. The input device 352 may also be operable to deactivate the disable or swimming mode, for example, when the user has finished swimming. In some embodiments, referring to FIG. 8, the external device 340 may include a user interface displaying a warning or notification that the swimming or disable mode is still active to remind the user to disable the mode, for example, if the user forgets to disable it after swimming. It is also contemplated that the external device 340 may provide haptic feedback (e.g., via vibrations) or emit an audible noise to remind the user to deactivate the mode. The valve assembly 310 may also deactivate Swim Mode automatically after a preset time limit, for example 8 hours. The valve assembly 310 may also deactivate Swim Mode automatically after a preset elapsed time period, for example one hour, in which no above-threshold ICP samples are taken. When deactivated, the controller 320 may control the valve 330 based on atmospheric pressure according to its normal function, for example, where the actual or unfiltered atmospheric pressure may be utilized to determine ICP. Additional uses for “Swim Mode” may include any time when very large transients in atmospheric pressure are anticipated. Examples may include skydiving, bungee jumping, ziplining, etc.

In some embodiments, it is contemplated that the input device 352 may also be operable to activate a mode to find their wearable device 342, for example, to activate an audible alarm or beacon to direct the patient to more readily locate a missing wearable device 342. In some embodiments, the external device 340 may communicate (via a notification) that the wearable device 342, and this notification may be sent to other external devices 340 or a host device (e.g., operated by the patient's physician). Technologies to implement wearable device 342 tracking include Bluetooth, Wi-Fi, the Global Positioning System, and the cellular Network. Examples of commercial devices that implement this feature include the ‘Find My iPhone’ app by Apple and the ‘Tile’ device by Life360.

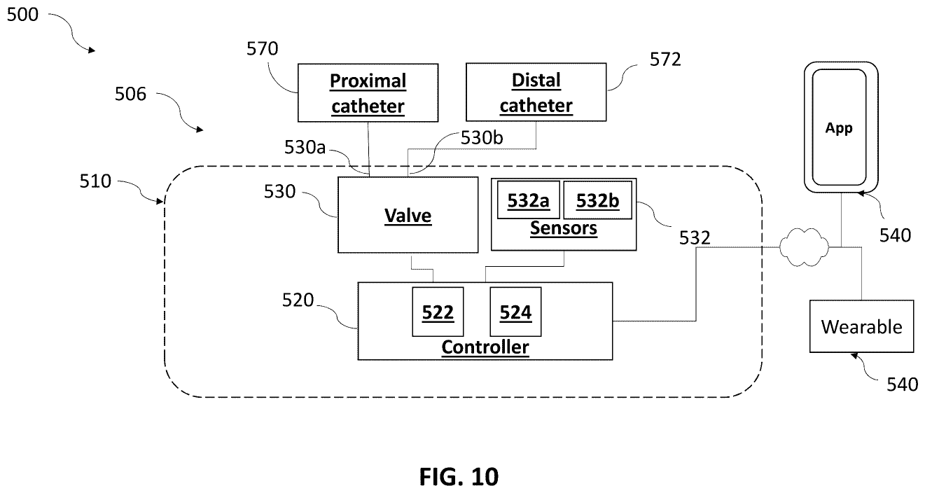

Referring now to FIG. 9, another example system 400 for treating hydrocephalus is shown. The system 400 includes a valve assembly 410 with a controller 420, a valve 430, a power supply 434, and one or more sensors 432 (e.g., a pressure sensor, tilt angle sensor, etc.) operatively connected to the controller 420. The controller 420 may embody a microcontroller that can send or receive data from the one or more sensors 432 to control an operation of the valve 430. The controller 420 may include a processor 422 and a storage device 424. The processor 422 may embody any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The storage device 424 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, non-volatile solid-state memory, etc.), unalterable memory (e.g., EPROMs), read-only memory, etc.). In some examples, the storage 424 includes multiple kinds of memory, particularly volatile memory and non-volatile memory. The storage device 424 may also embody a computer readable media on which one or more sets of instructions are embedded. The instructions may embody one or more of the methods or logic as described herein. In a particular embodiment, the instructions may reside completely, or at least partially, within any one or more of the storage device 424, the computer readable medium, and/or within the processor 422 during execution of the instructions.

The valve 430 is configured to connect to a distal catheter and a proximal catheter (not shown) to control the flow of cerebrospinal fluid (CSF) passing therethrough, for example, based on instructions from the controller 420, e.g., to open or close the valve assembly based on ICP data derived from the sensors 432, for example, ICP data derived from an absolute ICP, a tilt angle of the patient (e.g., the thorax or a catheter) relative to gravity, or atmospheric pressure (e.g., retrieved via the sensors 432 or via external devices communicatively coupled with the valve assembly 410).

The power supply 434 may be operatively connected to each of the valve assembly 430, the sensors 432, and the controller 420 to supply power thereto. The power supply 434 may embody a battery (e.g., a rechargeable high-density lithium coin cell). Wireless charging and power management circuitry may be included in the valve assembly 410 to ensure a safe, reliable recharge from an external charger device. In some embodiments, the valve assembly 410 may include a full “shutdown mode” that may be actuated to fully electrically disconnect the power supply 434 from the rest of the device during sterilization, shipment, or storage. In some embodiments, the valve assembly 410 may receive a wireless signal from an external device 240 to initiate shutdown. The system may place the valve 430 in a predetermined state just prior to shutdown, for example in the open state prior to sterilization (to facilitate gas diffusion throughout the fluid channel). The valve 430 shutdown state may be selectable by wireless software communication from the external device 240. The full shutdown command may require a security feature to limit shutdown capability to authorized personnel, for example factory or repair personnel. The security feature could be, for example, a password, an unusual input key sequence (“key dance”), or another means known in the art. In some embodiments, the valve assembly 410 may resume its normal operational state by holding a wireless charger (e.g., LTC 4124 made available by Analog Devices) near the implant power source 434 to wake up the valve assembly 410 and resume the implant's normal operational state.

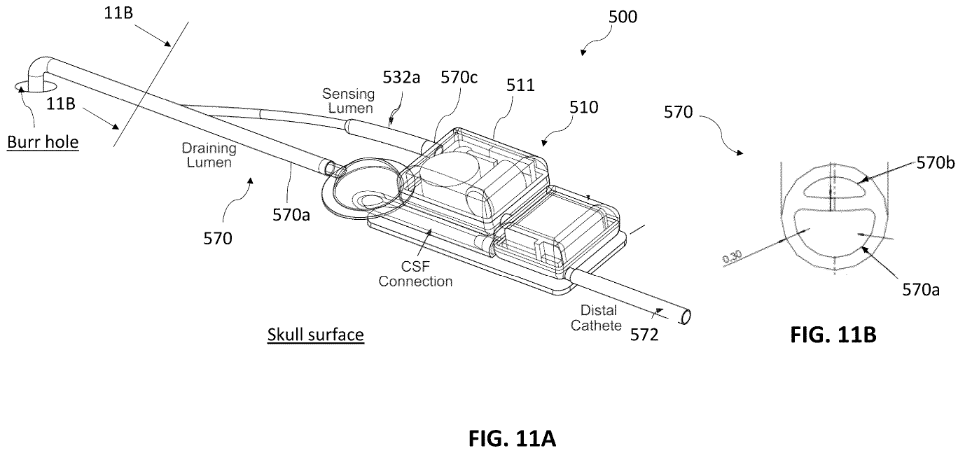

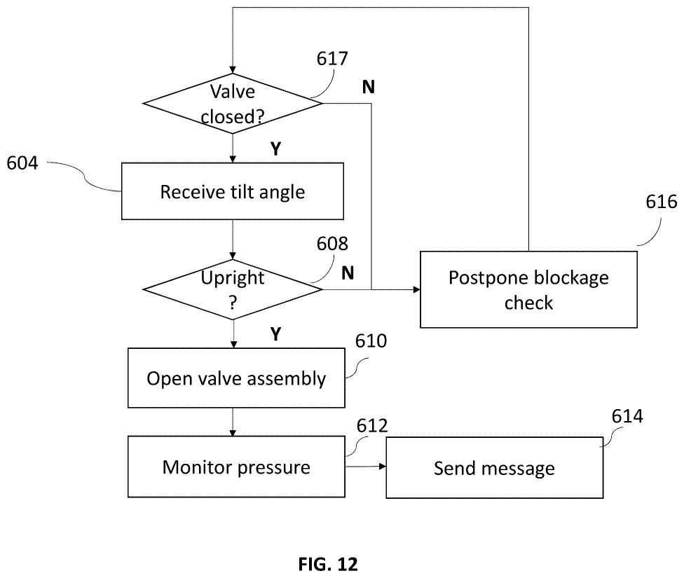

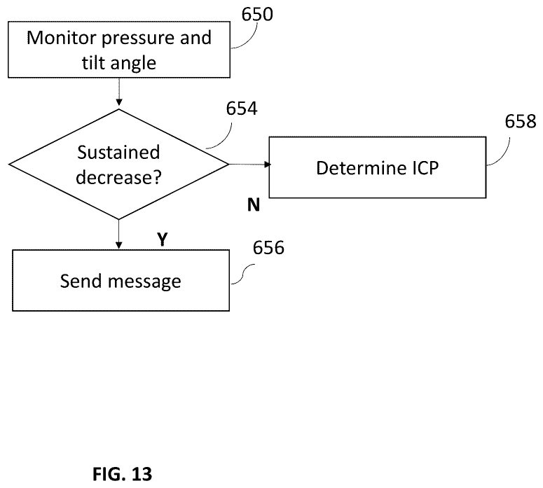

Referring to FIGS. 10-11B, another example system 500 for treating hydrocephalus is shown. The system 500 includes an implant 506 with a valve assembly 510, a proximal catheter 570, and a distal catheter 572. The valve assembly 510 includes a controller 520, a valve 530, and one or more sensors 532 operatively connected to the controller 520. The controller 520 may embody a microcontroller that can send or receive data from the one or more sensors 532 to control an operation of the valve 530. The controller 520 may include a processor 522 and a storage device 524. The processor 522 may embody any suitable processing device or set of processing devices such as, but not limited to: a microprocessor, a microcontroller-based platform, a suitable integrated circuit, one or more field programmable gate arrays (FPGAs), and/or one or more application-specific integrated circuits (ASICs). The storage device 524 may be volatile memory (e.g., RAM, which can include non-volatile RAM, magnetic RAM, ferroelectric RAM, and any other suitable forms); non-volatile memory (e.g., disk memory, FLASH memory, EPROMs, EEPROMs, non-volatile solid-state memory, etc.), unalterable memory (e.g., EPROMs), read-only memory, etc.). In some examples, the storage device 524 includes multiple kinds of memory, particularly volatile memory and non-volatile memory. The storage device 524 may also embody a computer readable media on which one or more sets of instructions are embedded. The instructions may embody one or more of the methods or logic as described herein. In a particular embodiment, the instructions may reside completely, or at least partially, within any one or more of the storage device 524, the computer readable medium, and/or within the processor 522 during execution of the instructions.

The valve 530 may include an inlet 530a operatively coupled to a proximal catheter 570 (e.g., a first catheter), and an outlet 530b operatively coupled to a distal catheter 582 (e.g., a second catheter). The valve 530 is configured to allow or block the flow of CSF from the inlet 530a to the outlet 530b based on instructions from the controller 520, e.g., to open or close the valve assembly based on ICP data derived from the sensors 532, where the data may include an absolute ICP, a tilt angle (e.g., the implant patient's thorax relative to gravity, an angle of a catheter relative to gravity), and atmospheric pressure (e.g., retrieved via the sensors 532 or via one or more external devices 540 (e.g., a smartphone or wearable device) communicatively coupled to the valve assembly 510.

The proximal catheter 570 may be configured to convey cerebrospinal fluid (CSF) to the inlet 530a of the valve 530, and the distal catheter 572 may be configured to convey the CSF from the outlet 530b thereof to another portion of the implant patient's body, e.g., into the abdominal peritoneum thereof.

The sensors 532 may include a pressure sensor 532a configured to measure intracranial pressure (ICP) in a subarachnoid space within the cranium, or within parenchyma of a patient's brain. The sensors 532 may also include a tilt sensor 532b configured to detect an angle of orientation of the implant patient's thorax relative to gravity, or another component of the system (e.g., the distal catheter) relative to gravity to determine whether the implant patient is in an upright position. In this manner, the pressure sensor 532a and the tilt sensor 532b may be respectively coupled to the controller 520 to transmit the ICP and tilt angle readings to the microcontroller, whereupon the controller 520 may include logic to determine an ICP based on the readings. In an embodiment, the pressure sensor 532a may be configured to measure ICP at a location outside the fluid channels defined by the first and second catheters.

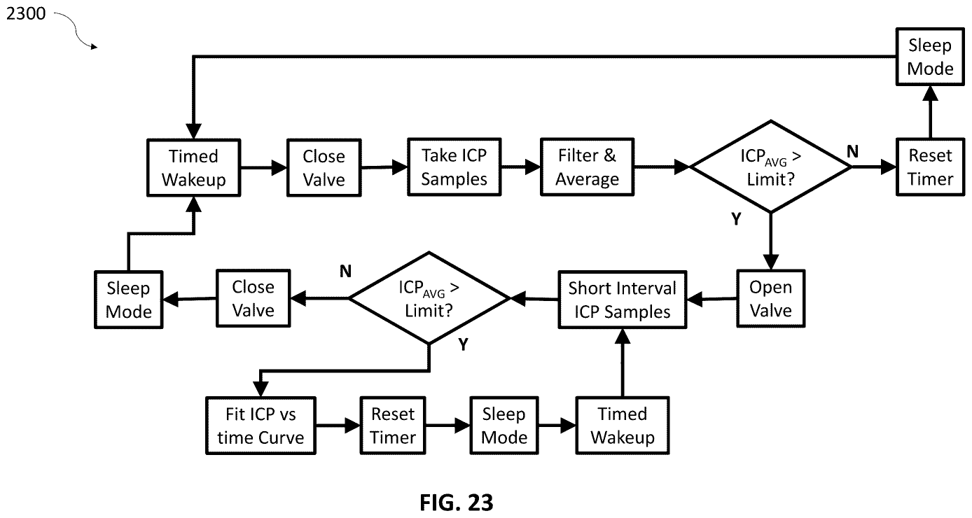

Referring to FIG. 11A, in some embodiments, the first catheter 570 may include a first lumen 570a and a second lumen 570b. The first lumen 570a (also referred to herein as a “draining lumen”) may define a fluid channel to drain CSF, and the second lumen 570b (also referred to herein as a “sensing lumen”) may define a fluid column to communicate ICP to a pressure sensor 532a, for example, an absolute ICP. The pressure sensor 532a may be disposed inside the implant housing 511 (for example, as part of the valve 530 (FIG. 10), or alternatively may embody a separate component in the housing 511 or be disposed at an end of the second lumen 570b proximate the housing 511. The pressure sensor 532a may, in another embodiment, be disposed as shown in FIG. 21. In this embodiment of the valve assembly 2100 the pressure sensor (not shown) may be housed in a capsule 2101 that may connect to implant valve assembly body 2103 via a flexible electrical or optical cable 2102. In alternative embodiments, the pressure sensor may be housed in a rigid rod or on a flexible wire (not shown) that extends downward from the implant valve assembly body 2103. The implant valve assembly body 2103 may rest subcutaneously on top of the patient's skull surface, and the capsule 2101 may feed through a burr hole drilled into the skull, and reside at least partially in the subarachnoid space or within the brain parenchyma. In an embodiment, the pressure sensor capsule may penetrate 5-10 mm below the brain's surface. The pressure sensor 532a may be cylindrical and may be mounted coaxially inside cylindrical capsule 2101. The sensitive surface of sensor 532a (not shown) may point downwards along the cylindrical axis. The capsule 2101 may have a solid bottom to preclude the brain parenchyma pushing against the sensitive surface and creating a false pressure reading. The capsule 2101 may have at least one open port or window 2104 on the side of the cylinder, near the sensitive surface, to allow cerebrospinal fluid to enter and contact the sensitive surface. The ports may be sized to allow fluid passage but keep brain matter from invaginating, clogging, or otherwise entering the capsule. The ports and capsule surface may be made of materials, and provided with geometries, sizes, coatings, surface features, or surface roughness/smoothness to inhibit fibrosis, inflammation, scarring, or other forms of biofouling. Note that unlike FIGS. 11A, 11B, and 12, FIG. 21 does not feature a dual lumen catheter. In this example, only a drain channel formed from the first lumen 570a is present. In this embodiment, the proximal catheter has only a single lumen and not a dual lumen construction as shown in the example of FIG. 11B.

Referring to FIGS. 11A, 11B, 12, and 21 the controller 520 may include logic to determine a possible blockage event in at least one of the first and second lumens 570a, 570b, or at the pressure sensor capsule port 2104 in FIG. 21. Specifically, the controller 520 may start the algorithm at a time when the valve is in the closed position (step 617). The controller 520 may receive a tilt angle reading from the tilt sensor 532b (step 604) to determine if the implant patient is in an upright position (step 608). If the implant patient is upright, then the controller 520 (at step 610) may open the valve 530 and monitor ICP (step 612) based on pressure readings from the pressure sensor 532a.

The controller 520 may monitor the ICP readings for a short period (e.g., between about 0.5 and 3 seconds in step 612) while the valve 530 is open to prevent over draining CSF. If the controller 520 receives pressure readings indicative of a large ICP drop due to fluid column weight (with the implant patient upright), then the controller 520 may determine that there is no blockage. The pressure drop may be nominally related to the torso size of the patient and can be between about 2 and 75 mmHg, or between about 5 and 50 mmHg, for example. The expected upright pressure drop for a given patient may be adjustable via an external device 540.

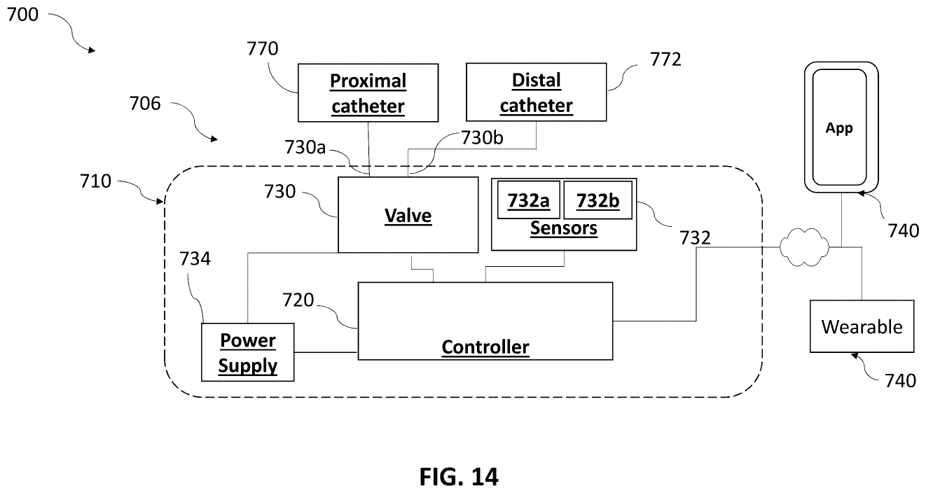

If the controller 520 receives pressure readings indicative of little or no ICP drop, then it may send an alert, alarm warning or message (step 614) to the implant patient via one or more of the external devices 540 communicatively coupled with the valve assembly 510, or to a host device (e.g., operated by the manufacturer of the implant, or the implant patient's physician). The message may indicate possible shunt obstruction or valve malfunction, and may advise the patient to contact their physician, or may refer the patient to a 3^{rd} party service or the manufacturer, who may take appropriate diagnostic or remediate action. In some embodiments, the controller 520 may postpone (step 616) a blockage check for another time, for example, at a time between about 10 hours and 24 hours after the check. This may be prompted by the controller 520 receiving tilt angle readings indicating that the implant patient is not in an upright position (at step 608), whereupon the controller 520 may reschedule the blockage check for a subsequent time. In some embodiments, blockage checks may be implemented automatically during a routine timed schedule, for example, at times the implant patient is most likely to be in an upright position, e.g., at 3 pm every day. In some embodiments, each check may be spaced out by at least 24 hours to avoid the possibility of over draining CSF. In some embodiments, a user may manually initiate a self-test wirelessly via external device 540. The blockage check may be performed based on a change or lack of change in absolute ICP reading when the valve opens; it therefore may not require obtaining an ambient pressure reading from an external device 540. In some embodiments, it is contemplated that the blockage check may be performed to detect blockage in the distal catheter, for example, for embodiments where the system utilizes a single lumen proximal catheter to drain CSF, and where the pressure sensor 532a is disposed within the valve assembly 510 (e.g., within the valve 530 thereof). In general, the blockage check may indicate one of several failure modes: drain channel obstruction (catheters 570 or 572, fluid channel or valve); sense channel obstruction (for dual lumen architecture); sensor capsule port 2104 obstruction or sensor biofouling (for capsule version in FIG. 21); sensor failure; valve 530 stuck in closed position; controller 520 failure; proximal catheter 570 (either lumen for dual lumen embodiment) detached from Valve Assembly fluid port 2105. We further note that the self-test algorithm of FIG. 12 further serves to exercise the valve 530 and prevent it from mechanically sticking in the closed position after long periods of time in that position. Valve exercise can be done as part of the self-test described here or as a separate exercise with no measurement. Valve exercise may be carried out automatically based on a timer that tracks time since last valve position change, or it may be manually initiated by a user via an external device 540. Frequency of tests may be limited to conserve battery power. The self-test algorithm may be carried out any posture—upright, recumbent, seated or postures in between these states—but that the measured change in ICP between valve open and valve closed may be most pronounced in the upright position. The results of these self-tests may be communicated to the patient or physician via the external devices 540 as described herein, may be logged, and may be input into artificial intelligence or machine learning algorithms that improve performance or use of the device. For example, an algorithm may monitor longitudinal self-test data and observe the ICP rate of change dP/dt that occurs when the valve is opened. If dP/dt for different tests decreases over time, it could indicate a gradual increase of fluid resistance in the fluid channel due to buildup of obstructive material. In this case, the system could issue an early warning or alert to the user. We further note that although exemplary architectures are described, the self-test concepts disclosed here apply to any architecture where the point of pressure sensing is outside the fluid drainage channel. Further, self-test or obstruction algorithms of this type are possible for system architectures that contain two pressure sensors, one upstream and one downstream of the valve. For these architectures, the system checks that pressure measurements P1 and P2 are approximately equal (within a preset threshold) with the valve is open, and unequal when the valve is closed, the inequality being more pronounced when the patient is upright. In other embodiments, it may not be necessary to open the valve solely for the purpose of detecting a possible obstruction, valve failure, proximal catheter detachment, or sensor failure. By monitoring ICP when the valve is open during normal operation, an algorithm may determine whether ICP decreases at or above a physiologically reasonable rate, the rate adjustable by the physician (typical values 0.1-5 mmHg per minute). If ICP does not change at this rate, there is a possible obstruction, valve, detachment, or sensor failure. Alternatively, the algorithm that uses normal valve operation may alert the user to a possible obstruction or failure when there has been insufficient ICP drop over a preset time limit when the valve is open.

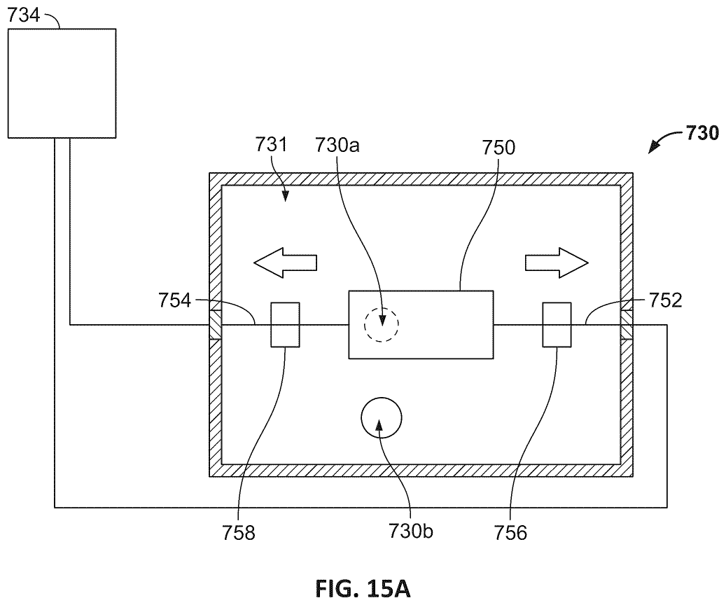

Referring to FIGS. 10, 11A and FIG. 13, in some embodiments, the controller 520 may include logic to determine whether the proximal catheter 570 (i.e., first catheter) is disconnected from the valve assembly 510 based on pressure communicated via the fluid column defined by the second lumen 570b. If the controller 520 receives pressure readings indicative of a sudden sustained pressure decrease (e.g., step 654), then it may send a message (step 656) to the one or more external devices 540 communicatively coupled to the valve assembly 510 indicating that the proximal catheter 570 could be disconnected, for example, external devices belonging to the implant patient, an implant patient family member, the manufacturer of the implant, or the implant patient's physician. If the controller 520 receives pressure readings indicating there is no sudden decrease, then it may continue with determining ICP (step 658) based on atmospheric pressure (e.g., provided by the external devices 540), absolute pressure (e.g., provided by the pressure sensor 532a), and a tilt angle (e.g., provided by the tilt sensor 532b to account for fluid column weight changes based on the posture of the implant patient).

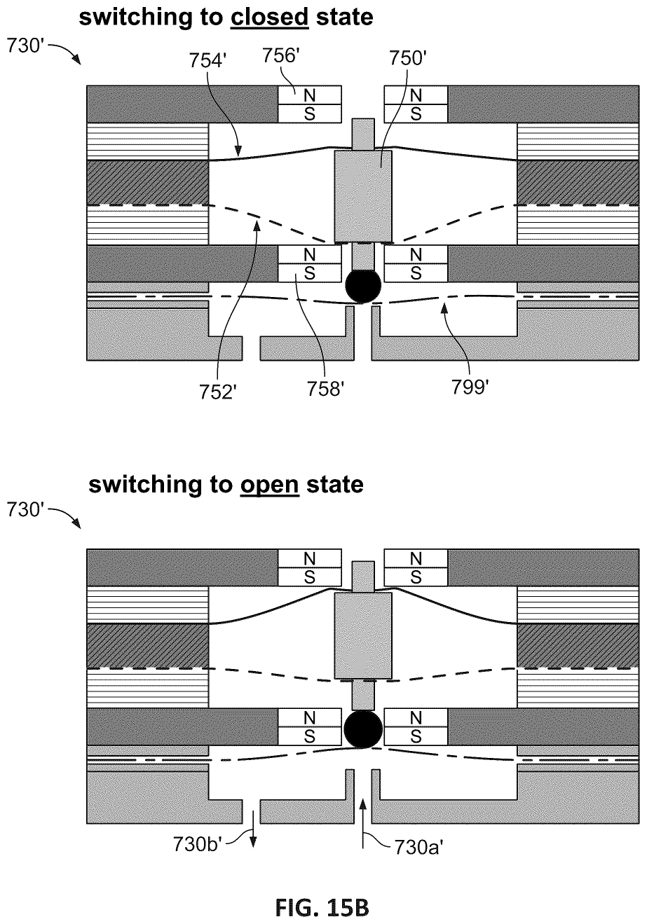

Referring to FIGS. 14-17, another example system 700 for treating hydrocephalus is shown. The system 700 includes an implant 706 with a valve assembly 710, a proximal catheter 770, and a distal catheter 772. The valve assembly 710 may include a controller 720, a valve 730, and one or more sensors 732 operatively connected to the controller 720. The valve assembly 710 may include a power supply 734 may be operatively connected to each of the valve 730, the sensors 732, and the controller 720 to supply power thereto. The power supply 734 may embody a battery (e.g., a rechargeable high-density lithium coin cell). Wireless charging and power management circuitry may be included in the valve assembly 710 to ensure a safe, reliable recharge from an external charger device.

The controller 720 may embody a microcontroller that can send or receive data from the one or more sensors 732 to control an operation of the valve 730. One or more external devices 740 may be communicatively coupled with the valve assembly 710 to receive or send data thereto, for example, atmospheric conditions (e.g., pressure, temperature, etc.).

The valve 730 may include an inlet 730a operatively coupled to a proximal catheter 770 (e.g., a first catheter), and an outlet 730b operatively coupled to a distal catheter 782 (e.g., a second catheter). Operatively, the valve 730 is configured to allow or block the flow of CSF from the inlet 730a to the outlet 730b based on instructions from the controller 720, e.g., to open or close the valve 730 based on ICP data derived from the sensors 732, where the data may include ICP (absolute ICP), a tilt angle of the implant patient (e.g., the implant patient's thorax relative to gravity, or a catheter relative to gravity), or atmospheric pressure (e.g., retrieved via the 732 or via one or more external devices 740 (e.g., a smartphone or wearable device) communicatively coupled to the valve assembly 710.

The proximal catheter 770 may be configured to convey cerebrospinal fluid (CSF) to the inlet 730a of the valve 730, and the distal catheter 772 may be configured to convey the CSF from the outlet 730b thereof to another portion of the implant patient's body, e.g., into the abdominal peritoneum thereof.

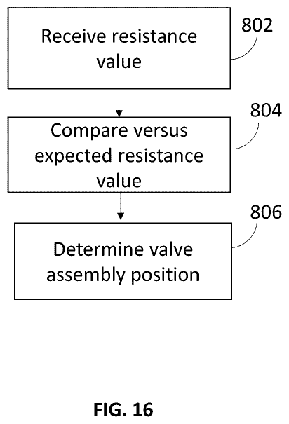

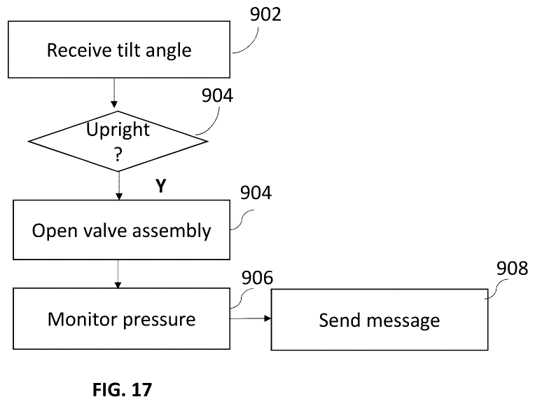

Referring to the examples shown in FIGS. 15A and 15B, the valve 730, 730′ may embody a shape memory alloy (SMA) actuated valve including a piston or plunger 750, 750′ a first actuator 752, 752′ and a second actuator 754, 754′ operatively connected to the plunger 750, 750′ and the power source 734. SMA actuated valves may be well suited for the implantable shunt as they are small in size, with low power draw, and feature very quiet operation. The plunger 750, 750′ may take on any suitable form, for example, a stainless steel plunger, a polymer-based plunger, a ferromagnetic material, or any suitable combination thereof. The plunger 750, 750′ is configured to sealingly close the inlet 730a of the valve 730, 730′ (adjoining a fluid channel that connects to the proximal catheter 770), for example, when the controller 720 determines that ICP is below a threshold value to preclude CSF from flowing into the valve 730, 730′, i.e., to prevent draining CSF. The upper sketch in FIG. 15B shows the valve in its closed position, with plunger 750′ (illustrated with a ball at a tip thereof) pushing a flexible membrane 799′ downwards to block the inlet 730a of the valve 730′.

In an embodiment, the valve assembly 710 incorporates the plunger 750, 750′ that is actuated by, for example, the SMA elements including the actuators 752, 752′, 754, 754′ to control CSF flow through the system. When the controller 720 determines, based on determined ICP and other sensor data, that drainage is required, the controller 720 applies current to the appropriate SMA actuator to move the plunger 750, 750′ from the closed position to the open position, thereby allowing CSF to flow from the proximal catheter 770 to the distal catheter 772. Conversely, when the ICP falls below a preset threshold, the controller 720 actuates the opposing SMA element to return the plunger 750, 750′ to the closed position, where it forms a fluid-tight seal against the valve inlet and prevents further CSF drainage. The use of magnets 756, 756′, 758, 758′ adjacent to the open and closed positions of the plunger 750, 750′ provides a latching mechanism that maintains the plunger 750, 750′ in the desired state with minimal power consumption. With this construction, the valve assembly 710 ensures precise, reliable, and energy-efficient regulation of CSF flow, while the unidirectional action of the plunger 750, 750′ prevents retrograde flow and protects against backflow-related complications. In this manner, the valve assembly 710 includes a one-way valve that allows CSF flow through the valve assembly from the intracranial space of the patient, and blocks CSF flow through the valve assembly 710 toward the intracranial space of the patient.

The plunger 750, 750′ is also configured to move to an open position (e.g., see bottom of FIG. 15B) to open the inlet 730a′, thereby fluidly connecting the inlet 730a′ with the outlet 730b. The first actuator 752, 752′ and the second actuator 754, 754′ may embody a strip or wire of SMA material that contracts when heated via electrical current provided by the power source 734, pulling the plunger 750, 750′ to one of two positions. Specifically, when the first actuator 752, 752′ is heated, it will contract, thereby pulling the plunger 750, 750′ to the open position (e.g., to the right in FIG. 15A), exposing the inlet 730a, 730a′ of the valve to enable CSF drainage. When the second actuator 754, 754′ is heated, it will contract and pull the plunger 750, 750′ (e.g., downwards in FIG. 15B), thereby closing the inlet 730a, 730a′ to prevent CSF drainage. In some embodiments, the valve assembly 730, 730′ may include a first magnet 756, 756′ and a second magnet 758, 758′ respectively disposed adjacent the first and second actuators 752, 752′ and 754, 754′. The magnets 756, 756′ and 758, 758′ may be operable to latch the plunger 750, 750′ to the respective opened and closed positions, for example, in those embodiments where the plunger 750, 750′ includes ferromagnetic material thereon, e.g., at opposing ends thereof. In this manner, when the plunger 750, 750′ is latched to a respective position, the power supply 734 may terminate the supply of electrical current to the corresponding actuator. For example, if the plunger 750, 750′ is pulled toward the closed position (e.g., see top of FIG. 15B) via the second actuator 754, 754′, it will become attracted to the second magnet 758, 758′ whereupon the controller 720 may control the power source 734 to terminate the supply of current to the second actuator 754, 754′. When the second actuator 754, 754′ is cooled, it may be deemed to be in a “low stress state,” and the first actuator 752, 752′ may be deemed to be in a “high tension state.” It should be understood that the first actuator 752, 752′ may be operable in a similar manner as the second actuator 754, 754′ to pull the plunger 750, 750′ toward the first magnet 756, 756′ when it is desired to open the valve assembly 730, 730′ (e.g., to expose the inlet 730a, 730a′ thereof). The first magnet 756′ may attract the plunger 750′ as it is pulled toward it via the first actuator 752, 752′ to a latched state, whereupon the controller 720 may control the power source 734 to terminate the supply of current to the first actuator 752, 752′. In this manner, the first actuator 752, 752′ (upon being cooled) may be deemed to be in a “low stress state,” and the second actuator may be deemed to be in a “high tension state.” In alternative embodiments, springs, mechanical latches, clips, or other mechanisms may be used instead of magnets, in combination with magnets, or in addition to magnets as a means of latching the valve. Latching is generally required to limit current draw to a brief burst during valve state transition, extending battery life and reducing thermal heating of the implant.

Each of the first and second actuators 752, 752′ and 754, 754′ may have a different electrical resistance when in the high tension state and when in the low stress state (i.e., not stretched).

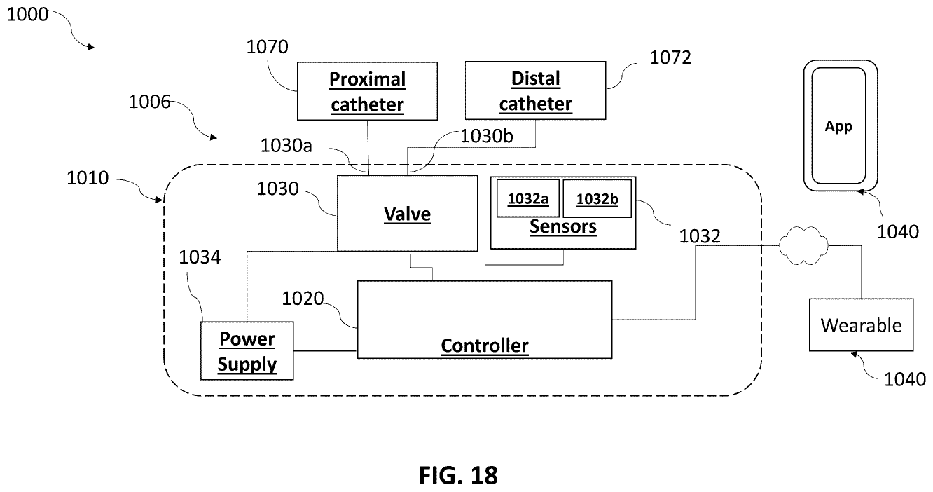

In this manner, the controller 720 may include circuitry to measure the resistance values (step 802 in FIG. 16), to determine whether the corresponding actuator 752, 752′ and 754, 754′ is in the high tension state or the low stress state, thereby enabling the controller 720 to determine the valve 730, 730′ position, e.g., whether it is opened or closed. For example, if the controller 720 receives electrical resistance values indicating that the first actuator 752, 752′ is in the low stress state and the second actuator 754, 754′ is in the high tension state, it may determine that the valve 730, 730′ is in an open position, where the inlet 730a, 730a′ is fluidly connected to the outlet 730b, 730b′ of the valve 730, 730′. Alternatively, the controller 720 may receive electrical resistance values indicating that the first actuator 752, 752′ is in the high tension state, and the second actuator 754, 754′ is in the low stress state, to determine that the valve 730, 730′ is in a closed position. The circuitry associated with Controller 720 may consist of any of the well-known methods for measuring resistance, for example detecting voltage across the actuator when a tightly controlled constant current of known value is passed through it. In other embodiments, a known voltage may be imposed across the actuator and the current resulting current through the actuator measured with methods well known in the art. In all cases, the amount of current being passed through the actuator should be small to conserve power and to prevent the actuator from changing its mechanical properties significantly during the measurement. It may be necessary for the processor to measure elapsed time or internal temperature of the valve assembly 710 to ensure that any residual heat from the last switching event has had time to dissipate, as temperature can affect the resistance of the SMA material and give an inaccurate resistance reading. Other methods, such as using the SMA actuator as the gain resistor in an operational amplifier circuit, are known in the art. Further methods, such as building the two SMA actuators into the well known Wheatstone Bridge configuration and measuring the delta between the midpoints of each bridge leg to determine which actuator is in tension and which is low stress. For typical SMA actuators, the difference in resistance between the high tension and low stress states may be between 5 and 200 milliohms.

In some embodiments, the controller 720 may determine whether each of the first and second actuators 752, 752′ and 754, 754′ are in the high tension state or the low stress state (to determine the valve assembly position) based on a comparison (step 804) of the corresponding electrical resistance values relative to expected or reference electrical resistance values, for example, resistance values obtained during factory calibration (e.g., accessible from a storage device of the controller 720). In this manner, the controller 720 (step 806) may determine the valve assembly position based on the comparison of electrical resistance values. In some embodiments, it is contemplated that the system 700 may utilize an artificial intelligence or machine learning algorithm to track trends in electrical resistance throughout time and adjust the expected resistance values.

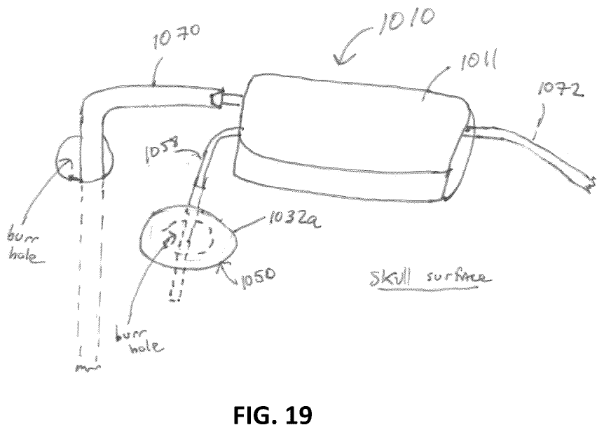

In some embodiments, referring to FIG. 17, the controller 720 may determine whether the valve 730 is opened or closed based on ICP and tilt angle measurements provided to the controller 720 via the sensors 732 (e.g., via the pressure sensor 732a and tilt sensor 732b). For example, if the controller 720 receives input indicating that the implant patient is in an upright position (steps 902 and 904), the controller 720 may briefly open the valve 730 (step 904), for example, for a period between about 0.5 seconds and 2 seconds, and monitor the ICP (step 906) to determine whether there is a pressure drop (e.g., due to fluid column weight in the ICP reading) during the time when the valve is open. In this manner, the controller 720 can confirm that the valve 730 is operating according to its normal function based on data indicating a pressure drop. Conversely, if the data indicates that there was no pressure drop, the controller 720 may send a message (step 908) or warning to the user, a host device (e.g., operated by the manufacturer of the implant), or the wearer's physician indicating a potential issue with the valve 730 that requires further investigation. This valve state verification may be carried out in any posture, but will yield the most obvious and reliable results in the upright position where there will be maximum difference between the valve open and valve closed ICP readings. This valve state verification algorithm may be carried out as part of the self-test and valve anti-sticking exercise functions described elsewhere in this application.

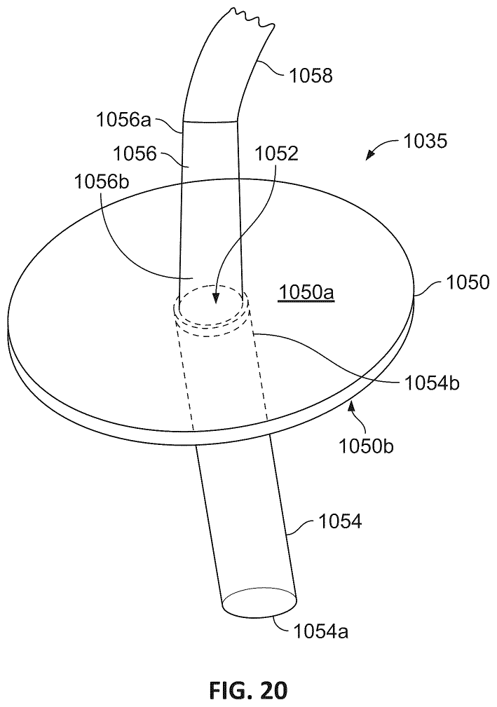

Referring to FIGS. 18-20, another example system 1000 for treating hydrocephalus is shown. The system 1000 includes an implant 1006 with a valve assembly 1010, a proximal catheter 1070, and a distal catheter 1072. The valve assembly 1010 includes a controller 1020, a valve 1030, and one or more sensors 1032 (e.g., a pressure sensor 1032a, tilt angle sensor 1032b) operatively connected to the controller 1020. The valve assembly 1010 may include a power supply 1034 operatively connected to each of the valve 1030, the sensors 1032, and the controller 1020.

The controller 1020 may embody a microcontroller that can send or receive data from the one or more sensors 1032 to control an operation of the valve 1030. One or more external devices 1040 may be communicatively coupled with the valve assembly 1010 to receive or send data to the controller 1020, for example, atmospheric conditions (e.g., atmospheric pressure, temperature, etc.) to determine gauge ICP.

The valve 1030 may include an inlet 1030a operatively coupled to a proximal catheter 1070 (e.g., a first catheter), and an outlet 1030b operatively coupled to a distal catheter 1072 (e.g., a second catheter). Operatively, the valve 1030 is configured to allow or block the flow of CSF from the inlet 1030a to the outlet 1030b based on instructions from the controller 1020, e.g., to open or close the valve 1030 based on ICP relative to a predetermined threshold value. The instructions may be based on ICP data derived from readings of the sensors 1032, where the readings may include ICP (absolute ICP) provided by the pressure sensor 1032a, a tilt angle provided by the tilt angle sensor 1032b (e.g., measuring an angle of the implant patient's thorax relative to gravity, or the angle of a catheter relative to gravity), or atmospheric pressure (e.g., provided by the sensors 1032 or via one or more external devices 1040 (e.g., a smartphone or wearable device) communicatively coupled to the valve assembly 1010.

The proximal catheter 1070 may be configured to convey cerebrospinal fluid (CSF) to the inlet 1030a of the valve 1030, and the distal catheter 1072 may be configured to convey the CSF from the outlet 1030b thereof to another portion of the implant patient's body, e.g., into the abdominal peritoneum thereof.

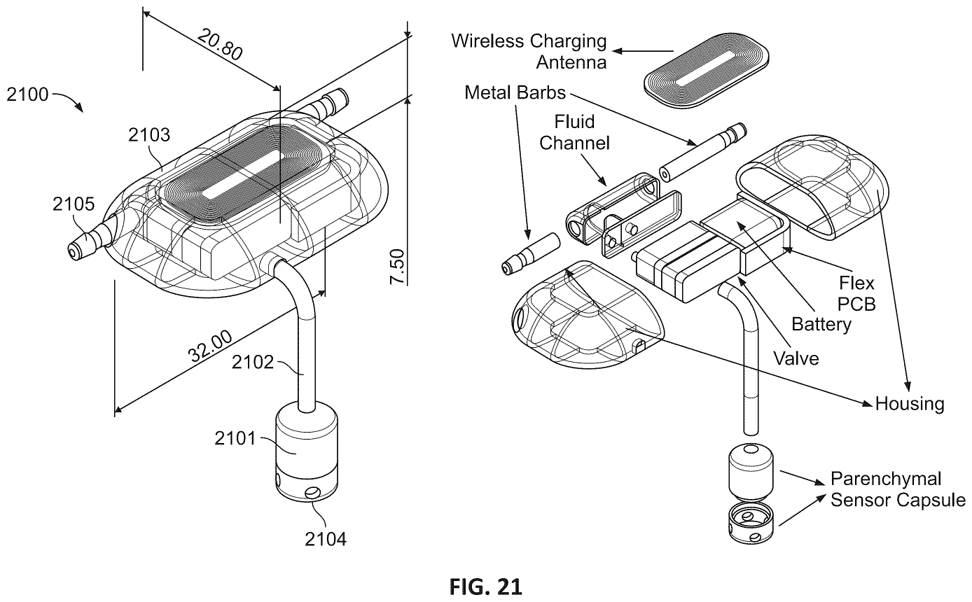

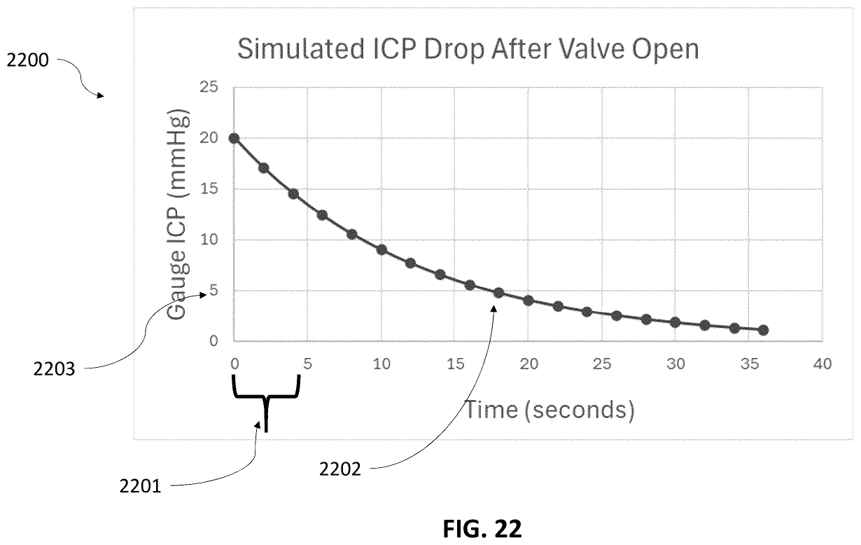

Referring to FIGS. 19-20, a pressure sensing assembly 1035 may be operatively connected to the pressure sensor 1032a to communicate pressure (ICP) to the pressure sensor 1032a. In the embodiment shown, the pressure sensing assembly 1035 includes a body 1050 with an upper surface 1050a and a lower surface 1050b. The lower surface 1050b is configured to rest on an outer surface of the skull. In the illustrated embodiment, the body 1050 is disposed over a burr hole in the skull and is dimensioned such that a diameter thereof is greater than the burr hole. In this manner, the body 1050 is dimensioned to be seated over the burr hole and extend past an outer periphery of the burr hole. In the embodiment shown, the body 1050 resembles a disc-shaped element. It is contemplated that the body 1050 may take on other shapes and forms, for example, a square or rectangular-shaped body. In some embodiments, the body 1050 may be made from biocompatible metal (examples include titanium and stainless steel), ceramic or glass, or plastic (examples include medical grade FKM, silicone, PEEK, or other polymers) or other material suitable for use in the implant patient.

An opening 1052 (FIG. 20) extends between the upper surface 1050a and the lower surface 1050b. A conduit 1054 extends from the lower surface 1050b and defines a fluid column extending between a distal end 1054a thereof (defining an inlet for CSF) and a proximal end 1054b thereof connected to the opening 1052. The fluid column is configured to communicate ICP from a space beneath the skull surface (e.g., in the subarachnoid space below the dura, or deeper in the parenchyma) to the pressure sensor 1032a. In some embodiments, the pressure sensor 1032a may be disposed in the pressure sensing assembly 1035, for example, at or near a proximal end 1054b of the first rod 1054 or in the body 1050, instead of being disposed in the valve assembly 1010.

A second conduit 1056 may extend from the upper surface 1050a with a proximal end thereof connected to the opening 1052, and a distal end connected to a flexible conduit 1058 (defining a flow channel) connected to the valve assembly 1010, for example, to the pressure sensor 1032a in those embodiments where the pressure sensor 1032a is disposed in the valve assembly 1010. In some embodiments, some or all of the first rod 1054, the second rod 1056, and the flexible conduit 1058 may include an electronic cable configured to transmit information (e.g., a measured ICP) to the controller 1020 from a pressure sensor in pressure sensing assembly 1035, for example, in those embodiments where the pressure sensor forms part of the pressure sensing assembly 1035 instead of being disposed in the valve assembly 1010.

In some embodiments, the connections between the first conduit 1054, the second conduit 1056, the body 1050, and the flexible conduit 1058 are waterproof and may be hermetically sealed. In some embodiments, the first conduit 1054 and the second conduit 1056 may be integrally formed with the body 1050, e.g., via plastic injection., machining, welding, adhesive, or other manufacturing processes known in the art. It is contemplated that the first rod 1054 and the second rod 1056 may be separate elements connected to the body 1050.

An example torso tilt sensor will now be described. In some embodiments, a tilt sensor (accelerometer or other type) may be embedded in the patient and configured to measure patient posture, in order to compensate for ICP measurement errors due to posture or to close the valve in the event of a sudden posture change that could cause a siphoning event. In some embodiments, the tilt sensor may be located inside the valve assembly (e.g., 110 in FIG. 2) that resides on the patient's skull surface. However, in certain cases, this location may be suboptimal because the tilt (angle between the sensor and the gravity vector) of the patient's head may be different than the tilt of the patient's torso, and the torso contains the distal catheter whose fluid column is the cause of the siphoning effect. To ensure tilt measurement of the torso and not just the head, several implant architectures may be implemented. For example, a tilt sensor may be placed in the distal catheter below the patient's neck line. And the tilt sensor may be wirelessly coupled to the electronics module on the skull, or may connect via wires mounted on the inside surface, the outside surface, or alongside the distal catheter. The wires may connect to the module via a connector, screws, or insulation penetrating connection as are known in the art. Second, a tilt sensor may be connected to the valve assembly (e.g., 110 FIG. 2) via a tethered wire and capsule, similar to the tethered ICP sensor described elsewhere in this application. This tethered tilt sensor would be separate from the distal catheter and would only go a short distance into the tunnel created for the distal catheter, just below the neckline. Third, a wireless tilt sensor with its own battery module may be placed subcutaneously in the torso at a safe and convenient location. Fourth, the tilt sensor may be a wearable that attaches to the torso externally with a “this end up” indicator, similar to a Zio® cardiac monitor by iRhythm, Inc, San Francisco, CA. Such a wearable may be disposable. Other embodiments may contain a torso tilt sensor as well as a head tilt sensor, to carry out ICP measurement correction (e.g. using the head sensor with valve open or closed) or anti-siphoning (e.g. using either or both tilt sensors with the valve open).

An example modular user interface will now be described. In some embodiments, the external device (e.g., the wearable device 142 in FIG. 1) may be configured as a small module with no direct user interface. The module may contain the communications, ambient pressure measurement, and processing functions described elsewhere in this and the reference applications. The wearable module may also contain a rechargeable battery and power management system. The wearable module may be further configured to mechanically couple to one or more wearable user interfaces, each of which would feature a different look and feel, and provide a different user interface appropriate for the age and needs of the patient. For example, the wearable user interface for an infant or toddler may be configured as a simple strap to go around the leg, and the wearable device could wirelessly communicate ICP and other system data to a caregiver's (e.g. parent's) smartphone, the smartphone providing the displays and controls needed to communicate with the implant, cloud server, etc. In another example, the same wearable user interface for an older child may include a necklace or wristwatch with a display that the user could view and operate. In yet another example, the wearable user interface for an adult may include a clip that clips into a pocket or under the clothing so as to not be visible, and communicates to the user's smartphone. In all cases, the same wearable device with no display or control may be connectable to the different wearable user interfaces as the patient grows or needs change.

Example algorithms for optimal valve operation will now be described. In some embodiments, it may be desirable to minimize power drain on the power supply/battery of the implant (e.g., 134 in FIG. 1) to minimize battery size and the frequency of battery recharge cycles. Since valve switching may represent a significant power draw on the battery, it may be advantageous to develop algorithms that minimize the number of valve transitions necessary for operation. It may further be advantageous to maximize the amount of time the implant is in its low-power sleep mode, and to generally minimize the “on” time for all system functions. In another embodiment, the implant may be devised to minimize or eliminate overdrainage of CSF from the patient's cranium during operation. In general, this means monitoring ICP frequently or continuously when the valve is in the open state, so that the valve can be closed immediately when an ICP below the preset threshold has been reached. But, frequent monitoring requires increased power draw, so it is desirable to configure the implant with operational algorithms that minimize both the risks of over drainage and excessive power draw.

In some embodiments, the implant may be configured to operate in a timed reading cycle where the implant is in low-power sleep mode for a period during which only an on-board timer is operating. The implant may then wake up, close the valve (if open), and take an ICP reading over a pre-set time period, nominally 10 seconds with a 50 Hz sampling rate (500 samples). Samples above or below preset thresholds may be removed and the remaining samples averaged to obtain a single absolute ICP reading, which may be converted to gauge ICP after subtracting the ambient Pressure reading received from the external device (e.g., wearable device). It may then compare the gauge ICP to the preset thresholds, set the valve state (open or closed) accordingly, pass data to the wearable device, and go back to sleep. Tilt readings may also be taken alongside the ICP readings for posture correction. The timed readings may have different wakeup intervals for the valve closed and valve open states. The intervals may be fixed and resettable by the physician wirelessly. The open state may have a shorter wakeup interval than the closed state in order to prevent over drainage. For example the closed state wakeup interval may be 15 minutes and the open state wakeup interval may be 30 seconds.

In an alternative embodiment, the system may remain in the awake mode for as long as the valve is in the open state, and may close the valve immediately when ICP measurements determine that the ICP is below the preset threshold. The system may use a moving window average (weighted or unweighted) when the valve is in the open state, and a simple average over a preset time window when the valve is in the closed state. To save power, the system may take ICP sample readings at a lower frequency when the valve is in the open state, for example 10 Hz. The system may apply power to the ICP sensor only for the time required to obtain a sample, and power off the ICP sensor in between samples. In this configuration, the system may not require a tilt sensor to determine whether a siphoning event may be occurring, as the rapid decrease in ICP may cause the valve to close immediately. Likewise, the moving average window used during open mode may have a shorter or longer duration than the than the single average window used during closed mode.

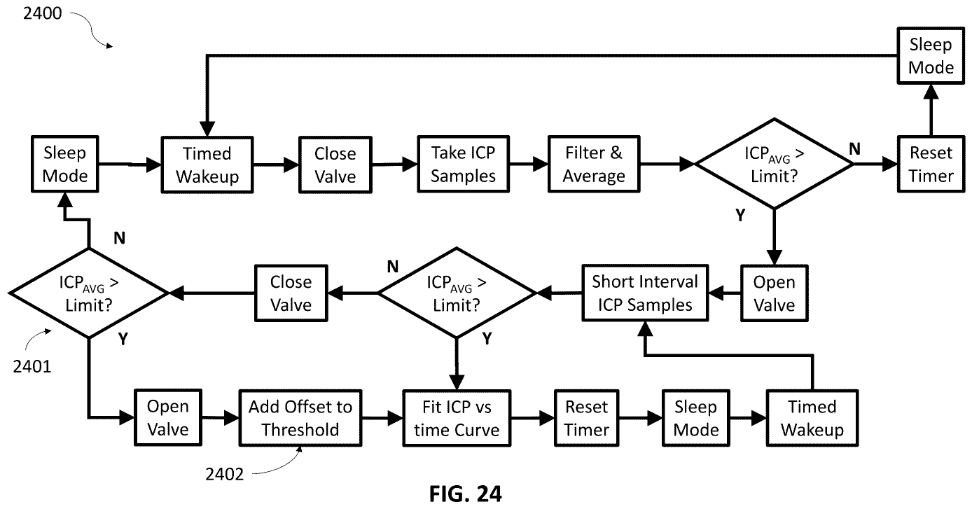

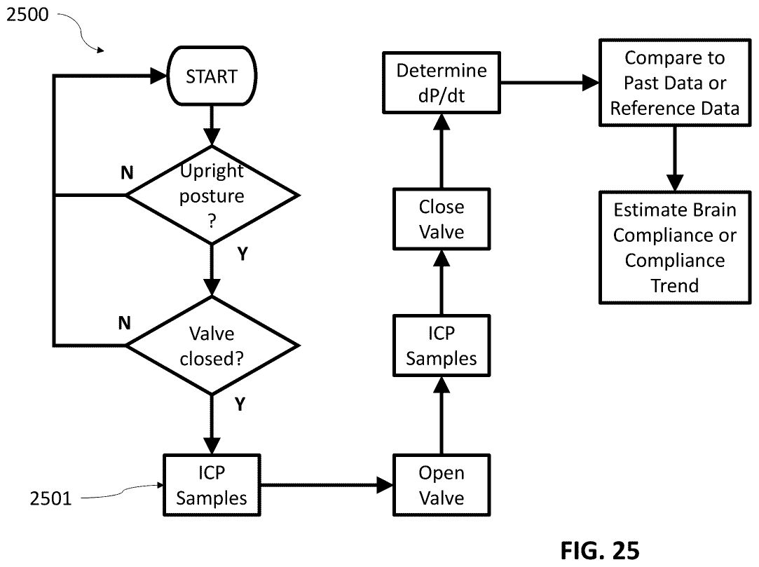

In other embodiments, the implant may take a small set of sample readings during valve open mode, and from these determine a time rate of change of ICP dP/dt. FIG. 22 is a graph 2200 of ICP over time after a the valve is opened, using exemplary data. The implant may sample ICP at regular intervals, in this example every 2 seconds, after the valve opens. After the four second interval 2201, the implant may have obtained a small number of samples, in the example three samples at times 0, 2 and 4 seconds. The implant may then stop sampling and perform a curve fit to an ICP vs time characteristic obtained by factory measurements or by an ideal curve; in this example the curve is a decaying exponential with formula y=Ae^{-kt}, where A is a magnitude coefficient, k is a decay rate, t is time, y is ICP and e is the base of the natural logarithm. Other characteristic curves are possible, for example nth order linear, or 1/x curves. The characteristic curve can be determined by benchtop characterization of the implant. For example, referring to FIG. 22, the implant's processor may then extrapolate the newly fit curve to determine the time 2202 at which the ICP will likely fall below the preset threshold 2203, which is 5 mmHg in this example. The implant's processor may then set the wakeup timer to wake up at, or shortly after, the time predicted by the curve at which ICP will fall below preset threshold 2203. The processor may then put the Implant into low-power Sleep mode, removing power from all functions except those necessary to count down the time interval. When the implant awakens again, it may take a new set of pressure samples. If these indicate that the ICP is below the preset threshold 2203, the processor may close the valve. If they indicate that ICP is above the preset threshold 2203, the processor may repeat the process of curve fitting to a short set of samples to set the next wakeup time. Flow chart 2300 in FIG. 23 depicts this process graphically.

To minimize valve transitions and save power, in some embodiments, different preset threshold values may be set for valve open and valve closed, the open threshold being at a higher pressure than the closed threshold. This may prevent oscillation of the valve at values near the threshold.

In some embodiments, an example methodology may prevent oscillations above and below the preset ICP threshold (that may occur between valve open and valve closed states), due to the fluid column pressure that is added to the ICP when the valve is open, using process 2400 in FIG. 24. This process is the same as that of FIG. 23, except that the method could require a second measurement of average ICP and comparison to the threshold limit in step 2401, after closing the valve. If closure of the valve increases ICP such that it exceeds the threshold limit, the valve may reopen, and the curve fit and interval setting may be performed once again (the ‘Y’ output from step 2401). This time, however, an offset may be added to the threshold limit in step 2402 to compensate for the fluid column increase, which may be determined by subtracting the most recent ICP readings between valve open and valve closed. The implant may go through this loop until the average ICP in step 2401, with valve closed, is below the threshold limit (the ‘N’ output from step 2401) and the system returns to sleep mode with valve closed.

The algorithms described here may be combined with a machine learning or artificial intelligence algorithm that retains historical data from the patient to use as training data for future readings. By comparing the accuracy of prediction of the time at which ICP will fall below the threshold limit against past readings, the device may adjust its curve fit formula to make future readings more accurate.

An example brain compliance estimation algorithm will now be described. In a further embodiment, the Implant may calculate the rate of change dP/dt from the curve in FIG. 22 when the valve switches from closed to open when the patient is in the upright position, and use it to estimate brain compliance, or to determine trends in brain compliance over time. Referring to FIG. 25, the compliance estimation algorithm may begin when the patient is in an upright posture (as determined by the tilt sensor) and the valve is closed. These conditions may yield the largest dP/dt results for comparison and may provide uniformity with previous measurements. In step 2501 the method could specify sampling the ICP sufficiently to determine a reliable average ICP while the valve is closed. The method may include opening the valve and further sampling the ICP. It is contemplated that a sudden drop in ICP may transpire due to the fluid column weight that has suddenly be added to the valve outlet. The valve may be left in the open position for as short a time as possible to prevent over drainage while still obtaining a reliable ICP sample, for example 300-500 milliseconds. The valve may be closed, and dP/dt may be determined based on the samples take before and after opening the valve. The new dP/dt may be compared to past data from the same patient, or reference data from literature or from the patient in a controlled setting. If dP/dt is higher than past data, then brain compliance may have decreased. If dP/dt is lower, compliance may have increased. These measurements may be used to estimate actual brain compliance or may be used to establish a relative compliance or trend.

The above algorithms may be combined with one another, or with previously disclosed algorithms. For example, the system may use a tilt sensor with a low power motion-activated wakeup signal, such as the ADXL363 by Analog Devices, Norwood, MA. When the patient is recumbent and the valve is open, the system may enable the motion-activated wakeup signal to monitor for sudden movement and may wake up the system to close the valve and execute an algorithm as in FIG. 23 or FIG. 24.

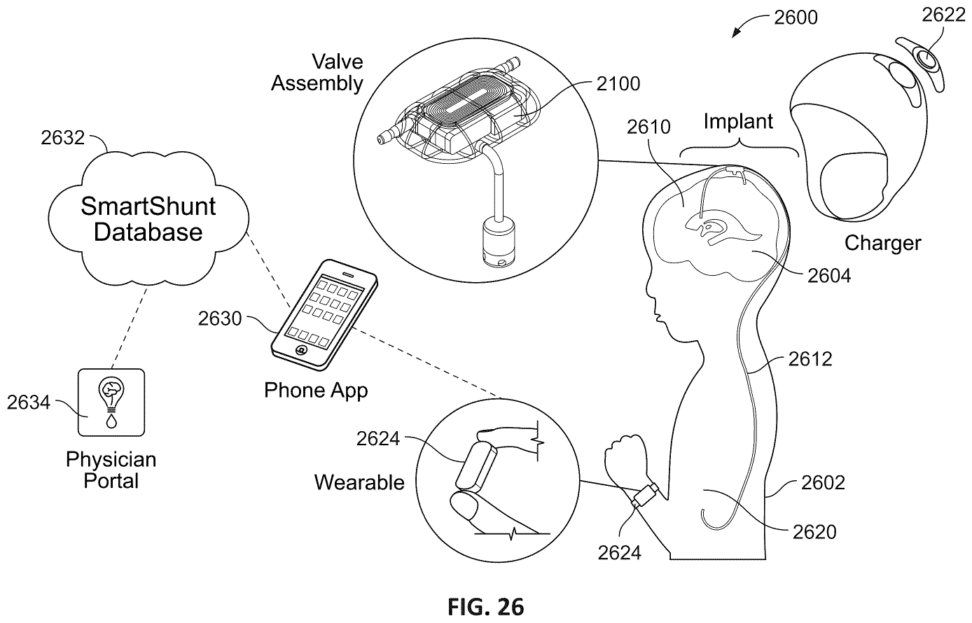

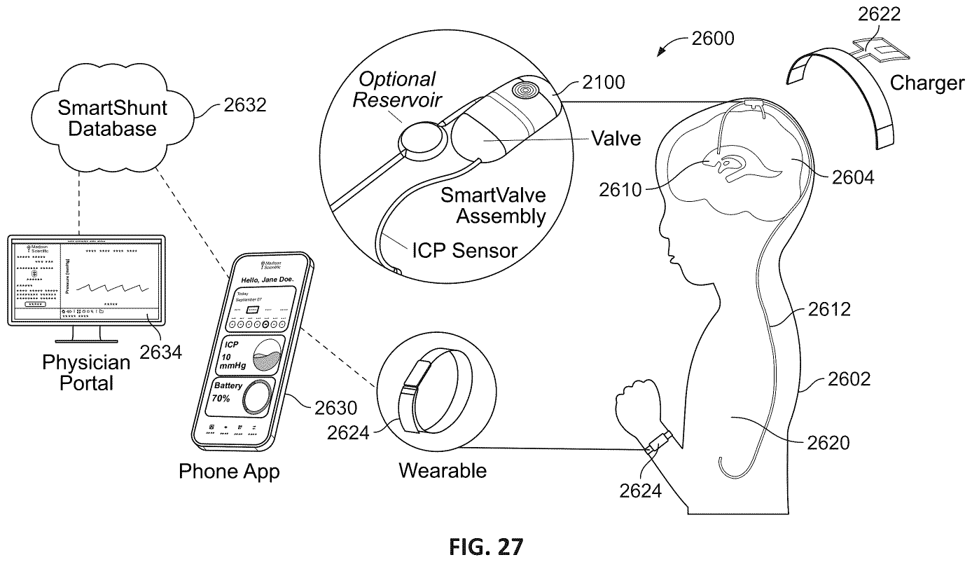

FIGS. 26 and 27 depict the valve assembly 2100 incorporated in an example system 2600 for treating hydrocephalus in a patient 2602. The system 2600 includes the valve assembly 2100, implanted in the patient 2602, where the controller and the one or more sensors monitor intracranial pressure (ICP) and other relevant data, including a tilt angle of the patient 2602. The valve assembly 2100 opens or closes to regulate the flow of CSF from an intracranial space 2604 through a proximal catheter 2610, the valve assembly 2100, and a distal catheter 2612, which drain and convey CSF from the intracranial space 2604 to another part of the body of the patient 2602, such as an abdominal peritoneum 2620. The valve assembly 2100 is powered by a battery, which can be recharged wirelessly using a wireless charger 2622, ensuring continuous operation without the need for invasive procedures to replace the battery.

A wearable device 2624 and a mobile device 2630 both communicate wirelessly with the valve assembly 2100 to provide real-time atmospheric data, receive updates on conditions of the patient 2602, and log event data. The wearable device 2624 and the mobile device 2630 may alert the patient 2602 or a clinician, for example, to low battery charge levels or valve malfunctions in the system 2600, and the mobile device 2630 can serve as a backup communication link to the valve assembly 2100. Data collected by the wearable device 2624 and the mobile device 2630 is transmitted to a cloud infrastructure 2632 or the remote server, where it is stored, analyzed, and used to make predictions about patient outcomes and implant performance. Clinicians and caregivers can access information on the cloud infrastructure 2632 through a remote terminal 2634 to monitor the conditions of the patient 2602, receive alerts about communication failures or potential issues, and make informed decisions about treatment for the patient 2602 remotely from the patient 2602. This integrated approach ensures continuous, real-time monitoring and rapid response to any changes in a condition or status of the patient 2602.

FIG. 26 depicts an embodiment of the system 2600 where the wireless charger 2622 is integrated into a soft, form-fitting EEG cap removably fixed to the head of the patient 2602, ensuring proper alignment between the wireless charger 2622 and the valve assembly 2100 for efficient wireless charging of the battery in the valve assembly 2100. In the depicted embodiment, the system 2600 also includes the wearable device 2624 as a clip that may be removably attached to clothing worn by the patient. FIG. 27 depicts an embodiment of the system 2600 where the wireless charger 2622 is supported on a flexible strap, which is also removably secured around the head of the patient 2602. In the depicted embodiment, the system 2600 also includes the wearable device 2624 as a bracelet that is worn directly on a wrist of the patient 2602. As such, the system 2600 may offer variations that enable customization and individual ease of use on a patient by patient basis in view of preference, comfort, and lifestyle choices while maintaining reliability in operation of the system 2600, including data communication between the valve assembly 2100, the wearable device 2624, the mobile device 2630, the cloud infrastructure 2632, or the remote terminal 2634.

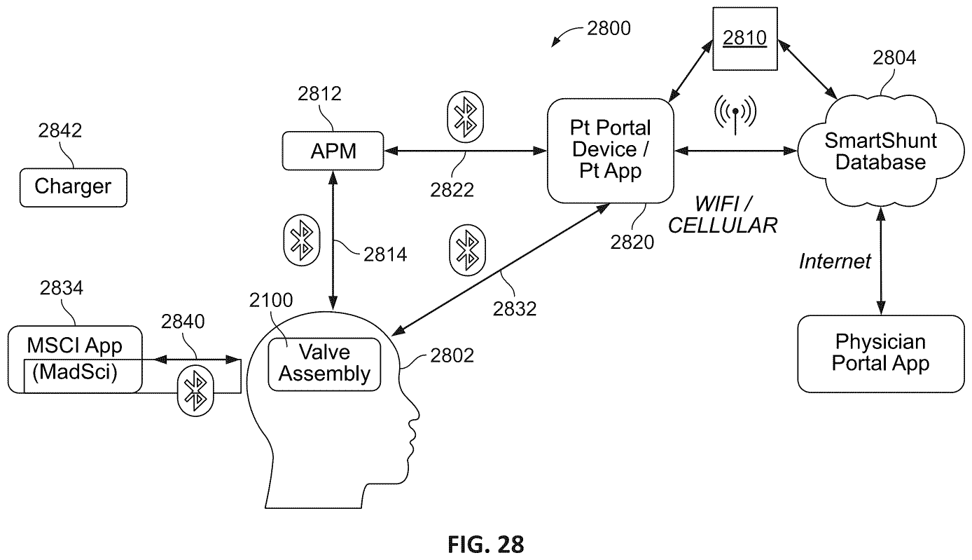

FIG. 28 depicts an embodiment of the valve assembly 2100 incorporated in an example system 2800 for treating hydrocephalus in a patient 2802 that employs a modular and tiered construction to data communication and device management. In this regard, the valve assembly 2100 as an implantable device or implant may lack cellular or wireless chipsets offering direct radio frequency (RF) communication capabilities to cloud-based databases, such as a cloud infrastructure 2804 or remote servers, such as a remote server 2810. With this construction, the valve assembly 2100 features relatively fewer parts implanted in the patient 2802, reducing a corresponding device size, complexity, and power consumption of the valve assembly 2100 in the patient 2802.

With continued reference to FIG. 28, the system 2800 leverages intermediary external devices, including an Ambient Pressure Monitor (APM) 2812 that facilitates data transfer, device management, and user interaction. The APM 2812, also referred to as the “wearable device,” is a compact, body-worn device that communicates with the valve assembly 2100 via Bluetooth Low Energy (BLE), indicated by an arrow 2814. The APM 2812 provides real-time ambient pressure data to the valve assembly 2100 over the BLE communication, which supports accurate ICP measurement and valve actuation by the valve assembly 2100, and also serves as a temporary data relay, storing operational and physiological data received from the valve assembly 2100. In embodiments, the APM 2812 does not require direct body contact with the patient 2802. In this regard, the APM 2812 communicates with the valve assembly 2100 wirelessly up to, for example, 10 feet from the patient 2802.

The system 2800 also includes a patient portal device 2820. In an embodiment, the patient portal device 2820 is a locked-down commercial smartphone or tablet, and configured to exclusively run a patient application. The patient portal device 2820 is equipped with cellular connectivity and serves as the primary conduit for uploading data to the cloud infrastructure 2804 or remote server 2810. The patient application is stored in memory on the patient portal device 2820 and executed by one or more onboard processors to carry out functions associated with device monitoring, data handling, and communication in the valve assembly 2100 and the patient portal device 2820. The patient application is configured to operate on the patient portal device 2820 at startup and to run persistently in the background, providing continuous monitoring functionality while preventing unauthorized or unintended use. The patient portal device 2820 is the primary conduit for uploading data, including intracranial pressure (ICP) readings and device status information, to the cloud infrastructure 2804 or the remote server 2810. The patient application also manages a user interface presented on the patient portal device 2820, optionally a touchscreen, through which the patient 2802 may view ICP readings, battery status, and system alerts associated with the valve assembly 2100, the APM 2812, or the patient portal device 2820. The patient portal device 2820, optionally via the application, is configured to block general-use features such as telephony, camera access, and web browsing. In this manner, the patient portal device 2820 may be dedicated exclusively executing functions through the patient application for medical data transmission and device management in the system 2800. With this construction, the system 2800 features enhanced security and operational reliability.

The APM 2812 periodically synchronizes information such as operational data and physiological data with the patient portal device 2820, also via BLE, indicated by an arrow 2822 to transfer all collected data. In an embodiment, the APM 2812 performs this synchronization at least once per day, ensuring that patient portal device 2820 maintains an up-to-date record of device and patient status in the system 2800. The patient portal device 2820 then uploads this data to the cloud infrastructure 2804 or the remote server 2810 using its cellular connection, indicated by arrows 2824, 2830. With this construction, the patient portal device 2820 enables remote monitoring by clinicians, data analytics, and long-term record-keeping, while maximizing reliability and user convenience.

In embodiments, the patient portal device 2820 communicates directly with the valve assembly 2100 via BLE, bypassing the APM 2812, indicated by arrow 2832 to provide ambient pressure data or relay operational data. Additionally, the system includes a MadSci Console Interface (MSCI) 2834 for advanced device management, diagnostics, or clinical oversight, typically accessed by clinicians or technical support personnel. The MSCI 2834 is a console interface that communicates directly with the valve assembly 2100 via BLE, indicated by an arrow 2840, bypassing the APM 2812 and the patient portal device 2820, to provide ambient pressure data to the valve assembly 2100 or relay operational data.

With continued reference to FIG. 28, the system 2800 includes a charger 2842 that may wirelessly recharge a battery in the valve assembly 2100. In an embodiment, the charger 2842 includes wireless communication capabilities, such as Bluetooth or power signal modulation, to facilitate data exchange with the valve assembly 2100 during charging, optimize charging efficiency, or relay charge status of devices in the system 2800. In further embodiments, the charger 2842 may function as a backup or substitute for the APM 2812 by performing redundant data relaying and ambient pressure monitoring functions.

As such, the system 2800 accommodates varying user preferences and lifestyles. In this regard, for example and without limitation, the patient 2802 may choose to carry only the APM 2812 during daily activities for convenience, leaving the patient portal device 2820 at home, or may bring the patient portal device 2820 along to access real-time ICP data or system alerts. In an embodiment, the patient 2802 is prompted to bring the APM 2812 and the patient portal device 2820 within BLE range, such a same room, at least once per day to synchronize data and maintain up-to-date records between devices in the system 2800.

The modular and tiered communication approach employed by the system 2800 offloads cloud communication from the valve assembly 2100 to external devices such as the APM 2812 and the patient portal device 2820. With this construction, the valve assembly 2100 may take relatively small, power-efficient, and less complex constructions. Further, the use of multiple external devices including the APM 2812, the patient portal device 2820, and the MSCI, with overlapping roles ensures redundancy and continuous operation in the system 2800, even if one such device is missing, malfunctioning, or otherwise unavailable. The locked-down nature of the patient portal device 2820 enhances security of the system 2800 by preventing unauthorized use or tampering, and the overall architecture allows the patient 2802 to tailor device usage to individual preferences while maintaining integrity and reliability of the system 2800.

In an embodiment, the valve assembly 2100 transmits a measured absolute intracranial pressure (ICP) value to the APM 2812 or the patient portal device 2820 via the wireless connection. Upon receipt, the APM 2812 or the patient portal device 2820 subtracts the ambient pressure, which it measures locally, to derive the gauge ICP of the patient 2802. The APM 2812 or the patient portal device 2820 then compares the calculated gauge ICP to a predetermined valve threshold and determines whether the valve assembly 2100 remains closed or is actuated open. Based on this determination, the APM 2812 or the patient portal device 2820 sends a corresponding valve command to the valve assembly 2100 over the wireless connection. As such, the APM 2812 or the patient portal device 2820 assumes the primary processing responsibilities and reduces a computational burden on a processor within the valve assembly 2100. In a further embodiment, the valve assembly 2100 transmits each individual pressure sample and accelerometer sample wirelessly to the APM 2812 or the patient portal device 2820. The APM 2812 or the patient portal device 2820 averages the received samples to obtain the average ICP and calculates a tilt offset to further refine the gauge ICP calculation. With this construction, the APM 2812 or the patient portal device 2820 further simplifies the processing requirements for the valve assembly 2100 and increases the volume of data transmitted between the valve assembly 2100 and the APM 2812 or the patient portal device 2820.

In embodiments, the system 2800 detects off-nominal conditions when at least one of the valve assembly 2100, the APM 2812, or the patient portal device 2820 identifies an anomaly in collected or received data, including operational or physiological data. Such an off-nominal condition may arise in the system 2800 from a deviation in expected physiological parameters, device performance, or communication status. For example, the valve assembly 2100 may detect an unexpected trend or abrupt change in intracranial pressure (ICP) readings, such as a sustained elevation or drop outside of preset thresholds, or a lack of expected ICP response following actuation of the valve assembly 2100. The APM 2812, the patient portal device 2820, or the MSCI 2834 may identify inconsistencies in ambient pressure measurements, irregularities in the frequency or content of data transmissions from the valve assembly 2100, or a failure to receive timely updates. The APM 2812, the patient portal device 2820, or the MSCI 2834 may further recognize anomalies such as missed data synchronization events, repeated communication failures between various devices in the system 2800, or operational data indicating abnormal device status, such as low battery warnings, sensor faults, or unexpected device resets. Notably, where the processor detects excessive timer resets in the system 2800 by a user or the APM 2812, the patient portal device 2820, or the MSCI 2834, the processor may determine the valve assembly 2100 to be in a potentially unsafe condition for the patient 2802.

Upon detection of an off-nominal condition, the detecting device, such as one of the APM 2812, the patient portal device 2820, or the MSCI 2834 generates a warning, alarm, or notification, and transmits a corresponding alert to the other devices in the system 2800, which may include the cloud infrastructure 2804 or the remote server 2810 for further analysis and clinician review. With this construction, the system 2800 supports a coordinated detection and reporting framework and enables rapid identification and response to potential device malfunctions, physiological events, or communication disruptions, thereby supporting patient safety and system reliability.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define embodiments disclosed herein. Accordingly, the present specification is intended to embrace all such alterations, modifications and variations that fall within the spirit and scope of the appended claims.

Furthermore, to the extent that the term “includes” is used in either the detailed description or the claims, such term is intended to be inclusive in a manner similar to the term “comprising” as “comprising” is interpreted when employed as a transitional word in a claim.

Also, as used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Additionally, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel.

1. A system for treating hydrocephalus in a patient, the system comprising:
a valve assembly comprising:
a controller that actuates the valve assembly between an open position and a closed position, wherein the valve assembly conveys cerebrospinal fluid (CSF) from an intracranial space of the patient in the open position;
one or more sensors operatively connected to the controller, wherein the one or more sensors generate internal sensor data indicating a condition of the patient, and the controller actuates the valve assembly based on the internal sensor data;
a battery operatively connected to the one or more sensors or the controller; and
an external device in wireless communication with the valve assembly, wherein the external device generates an audible or visual warning, alarm, or notification when:
an amount of charge in the battery is below a predetermined threshold,
the controller, the external device, or a remote server determine an event occurrence, an obstruction condition, or an off-nominal condition based on the internal sensor data,
a manual override of the valve assembly exceeds a predetermined duration, or
the external device attempts and fails to communicate with the valve assembly, wherein the one or more sensors comprise a first pressure sensor that generates the internal sensor data indicating an absolute intracranial pressure (ICP) of the patient, the external device comprises a second pressure sensor that generates external sensor data indicating an ambient pressure outside the patient, the controller derives a gauge ICP of the patient from the internal sensor data and the external sensor data, the controller actuates the valve assembly based on the gauge ICP and wherein the external device comprises a wearable device and a mobile device, and wherein the mobile device repeatedly attempts communication with the wearable device within a predetermined interval, determines whether the wearable device has communicated the external sensor data to the valve assembly based the attempted communication, and transmits the external sensor data to the valve assembly based on the determination.

2. (canceled)

3. The system of claim 1, wherein the one or more sensors comprises a tilt angle sensor coupled to the controller, wherein the tilt angle sensor generates orientation data indicating a tilt angle of the patient relative to gravity, and
wherein the controller applies a pressure offset to the internal sensor data indicating the absolute ICP based on preset calibration data associated with orientation to gravity, wherein the controller corrects a determined ICP pressure and derives the gauge ICP based on the corrected determined ICP pressure.

4. The system of claim 3, wherein the preset calibration data comprises a lookup table or formula associated with posture-dependent pressure offsets, and is determined noninvasively by recording ICP readings while the patient is sequentially positioned in different orientations relative to gravity, the orientations including upright, supine, prone, and lateral decubitus positions.

5. (canceled)

6. The system of claim 5, wherein the wearable device is smaller than the mobile device, and the mobile device has greater wireless communication range than the wearable device.

7. The system of claim 5, wherein the mobile device is a dedicated, single-purpose device operating on a locked operating system that suspends, constrains, or disables automatic updates.

8. The system of claim 5, wherein, in response to determining the wearable device has not communicated the external sensor data, the mobile device enters a beacon mode in which the mobile device periodically broadcasts the external sensor data to the valve assembly.

9. The system of claim 8, wherein, in response to failing to receive the external sensor data from the wearable device within a predetermined duration, the valve assembly initiates periodic wake cycles in which a wireless transceiver of the valve assembly is activated to receive the external sensor data from the mobile device, the wake cycles occurring at an interval shorter than an interval at which the mobile device periodically transmits the external sensor data in the beacon mode.

10. The system of claim 5, wherein the mobile device transmits the internal sensor data, the external sensor data, or operational data from the valve assembly or the wearable device to a cloud infrastructure or the remote server.

11. The system of claim 1, wherein the external device comprises a user interface operable to actuate transmission of external sensor data or to initiate or terminate a swim mode,
in response to activation of the swim mode, the valve assembly performs at least one of: initiating or modifying a timed reading cycle; transitioning to one of the open position and the closed position as a designated safe state; or disregarding pressure measurements that exceed an adjustable threshold between 5 mmHg and 20 mmHg, and
when the swim mode is engaged for a predetermined duration, the external device generates a reminder to terminate the swim mode or the valve assembly automatically deactivates the swim mode.

12. The system of claim 18, further comprising:
a first catheter operatively connected to an inlet of the valve assembly, wherein the first catheter conveys CSF from within the intracranial space toward the inlet; and
a second catheter operatively connected to an outlet of the valve assembly, wherein the second catheter conveys the CSF from the outlet to a location outside the intracranial space,
wherein the one or more sensors includes a pressure sensor located outside a flow path of the CSF defined by the first catheter and the second catheter, and the controller determines a possible blockage at the first catheter, the inlet, the outlet, the valve or the second catheter in response to the pressure sensor failing to detect a decrease in intracranial pressure in the patient at the second catheter after actuating the valve assembly toward the open position.

13. The system of claim 18, further comprising:
a first catheter operatively connected to an inlet of the valve assembly, wherein the first catheter conveys CSF from within the intracranial space toward the inlet through a first lumen and a second lumen, the first lumen and the second lumen are separate along the first catheter at the inlet, the first lumen conveys CSF from within the intracranial space toward the inlet, and the second lumen fluidly connects CSF from within the intracranial space to a pressure sensor located within the valve assembly; and
a second catheter operatively connected to an outlet of the valve assembly, wherein the second catheter conveys the CSF from the outlet to a location outside the intracranial space,
wherein the controller determines a blockage at the first lumen, the inlet, the outlet, or the second catheter as the event occurrence in response to the pressure sensor failing to detect a decrease in intracranial pressure in the patient at the second lumen after actuating the valve assembly to the open position.

14. The system of claim 1, wherein, in response to detecting an unsafe condition of the valve assembly, the controller actuates the valve assembly toward the open position based on the sensor data received from the one or more sensors most recently transmitted to the controller, prior to the battery being below the predetermined threshold, and wherein the valve assembly remains in the open position and performs passive CSF drainage from the intracranial space.

15. The system of claim 14, wherein the unsafe condition comprises at least one of:
a battery charge below a predetermined threshold;
excessive timer resets;
ICP or tilt readings outside of a predetermined threshold range;
missing ambient pressure data received from an external device after a predetermined time;
a temperature within the valve assembly outside a predetermined range;
a suspected valve setting error;
corrupted or ineffective data transmission; or
a command from an authorized external device to power down.

16. The system of claim 14, wherein the valve assembly comprises a flow restriction with a forward pressure drop having a predetermined value when the valve assembly moves toward a safe state in the open position.

17. The system of claim 16, wherein the valve assembly further comprises a one-way valve that forms the flow restriction when the valve assembly moves toward the safe state.

18. A system for treating hydrocephalus in a patient, the system comprising:
a valve assembly comprising:
a controller that actuates the valve assembly between an open position and a closed position, wherein the valve assembly conveys cerebrospinal fluid (CSF) from an intracranial space of the patient in the open position;
one or more sensors operatively connected to the controller, wherein the one or more sensors generate internal sensor data indicating a condition of the patient, and the controller actuates the valve assembly based on the internal sensor data; and
a battery operatively connected to the one or more sensors or the controller; and
an external device in wireless communication with the valve assembly, wherein the external device generates an audible or visual warning, alarm, or notification when:
an amount of charge in the battery is below a predetermined threshold,
the controller, the external device, or a remote server determine an event occurrence, an obstruction condition, or an off-nominal condition based on the internal sensor data,
a manual override of the valve assembly exceeds a predetermined duration, or
the external device attempts and fails to communicate with the valve assembly, wherein the external device includes a first wearable device and a mobile device or a second wearable device,
the first wearable device, the mobile device, or the second wearable device prompt the patient to activate both the first wearable device and the mobile device or the second wearable device at a same time, within a corresponding wireless communication range of the valve assembly, the first wearable device, and the mobile device or the second wearable device, after a predetermined period of time without detecting the activation, and
the valve assembly transmits the internal sensor data or first local operating data to the first wearable device and the mobile device or the second wearable device in response to detecting the activation,
the first wearable device and the mobile device or the second wearable device transmit external sensor data or second local operating data to each other and the valve assembly in response to detecting the activation, and
at least one of the first wearable device and the mobile device or the second wearable device generate an audible or visual warning, alarm, or notification when, in response to the activation:
an amount of charge in a power supply operatively connected with any of the valve assembly, the first wearable device, the mobile device, and the second wearable device is below a predetermined threshold,
the controller, the external device, or the remote server determines the event occurrence based on the internal sensor data and the external sensor data, the first local operating data, or the second local operating data, or
both the first wearable device and the mobile device or the second wearable device attempt and fail to communicate with the valve assembly.

19. The system of claim 18, wherein the valve assembly operates in a repeated cycle,
the valve assembly is inactive in a low-power sleep state for a predetermined time period during the cycle,
at an end of the predetermined time period, the valve assembly wakes up and advertises for the external device, wherein when the valve assembly connects to the external device, downloads ambient pressure data and new valve assembly settings from the external device, and updates internally stored settings with any new settings;
after the end of the predetermined time period, the valve assembly determines an absolute ICP of the patient, derives a gauge ICP by subtracting the ambient pressure data from the absolute ICP, compares the gauge ICP to an internally stored preset threshold, moves toward the open position upon determining the gauge ICP exceeds the preset threshold, uploads the gauge ICP, valve position, and battery level data to the external device, and returns to the low-power sleep state for the predetermined time period.

20. The system of claim 19, wherein the controller averages detected absolute ICP over a time window.

21. The system of claim 19, wherein the valve assembly is in the closed position when detecting the absolute ICP.

22. The system of claim 19, wherein the settings downloaded from the external device comprise one or more of a valve opening threshold pressure, a valve closing threshold pressure, a duration of the predetermined time period for averaging absolute intracranial pressure (ICP), a software update for the valve assembly, and a parameter specifying battery shutdown capacity or voltage.

23. The system of claim 19, wherein, during the cycle, when an external charger is applied to the valve assembly, the valve assembly pauses entry to the low-power sleep state, and remains awake between measurement and valve switching cycles.

24. The system of claim 19, wherein the predetermined time period is set based on a state of the valve assembly being in the open position or the closed position.

25. The system of claim 24, wherein the external device includes a user interface that actuates the valve assembly toward the open position or the closed position, obtains a determined ICP, temperature, battery, or tilt condition from the valve assembly, and actuate the valve assembly to shut down or enter an automatic mode.

26. The system of claim 19, wherein the controller causes the valve assembly to move toward the open position and enter a sleep mode after failing to communicate with the external device after a predetermined time period.

27. The system of claim 1, wherein the wearable device initially generates the warning, alarm, or notification including an audible sound having a first intensity, then generates the warning, alarm, or notification including an audible sound having a second intensity greater than the first intensity after a first predetermined period of time, and then generates the warning, alarm, or notification including a visible light after a second predetermined period of time following the first predetermined period of time.

28. A system for treating hydrocephalus in a patient, the system comprising:
a valve assembly comprising:
a controller that actuates the valve assembly between an open position and a closed position, wherein the valve assembly conveys cerebrospinal fluid (CSF) from an intracranial space of the patient in the open position;
one or more sensors operatively connected to the controller, wherein the one or more sensors generate internal sensor data indicating a condition of the patient, and the controller actuates the valve assembly based on the internal sensor data;
a battery operatively connected to the one or more sensors or the controller; and
an external device in wireless communication with the valve assembly, wherein the external device generates an audible or visual warning, alarm, or notification when:
an amount of charge in the battery is below a predetermined threshold,
the controller, the external device, or a remote server determine an event occurrence, an obstruction condition, or an off-nominal condition based on the internal sensor data,
a manual override of the valve assembly exceeds a predetermined duration, or
the external device attempts and fails to communicate with the valve assembly, wherein the battery is a first battery,
the external device comprises a second battery,
the external device generates the warning, alarm, or notification including an audible, visual, or haptic indication having a first intensity associated with a first amount of charge in the first battery or the second battery being below a first predetermined threshold,
the external device generates the warning, alarm, or notification including an audible, visual, or haptic indication having a second intensity greater than the first intensity and associated with a second amount of charge in the first battery or the second battery being below a second predetermined threshold lower than the first predetermined threshold, and
the external device generates the warning, alarm, or notification including a visible light associated with a second amount of charge in the first battery or the second battery being below a third predetermined threshold lower than the second predetermined threshold.

29. The system of claim 28, wherein the external device generates the warning, alarm, or notification repeatedly or continuously in response to detecting that the amount of charge in the first battery or the second battery is below the first predetermined threshold, the second predetermined threshold, or the third predetermined threshold, and discontinues generating the warning, alarm, or notification when the external device detects the first battery or the second battery is charging or has been recharged.

30. The system of claim 1, wherein the wearable device generates the warning, alarm, or notification including an audible sound, light, or haptic feedback,
and wherein the wearable device uploads the warning, alarm, or notification to the mobile device, and the mobile device displays text indicating the warning, alarm, or notification.

31. The system of claim 1, wherein the external device comprises a user interface that receives patient data,
the external device, the valve assembly, or the remote server correlate the patient data to ICP of the patient, and predict ICP based on the correlation, and
the controller actuates the valve assembly, or the external device generates the warning, alarm, or notification based on the prediction.

32. The system of claim 1, further comprising a remote server operatively coupled to the external device, and a remote terminal operatively coupled to the remote server,
wherein the external device transmits the internal sensor data, external sensor data from the external device, or operational data from the valve assembly or the external device to the remote server repeatedly within a predetermined interval, and
the remote terminal monitors the remote server for updates from the external device, and generates a warning, alarm, or notification when the remote terminal determines the external device has not communicated the internal sensor data, the external sensor data, or the operational data within the predetermined interval.

33. The system of claim 1, wherein the external device, the controller, or the remote server stores collected data including the internal sensor data, external sensor data, event data, operating data, biometric data, or clinical profile data associated with the patient, the valve assembly, or the external device,
the external device, the controller, or the remote server correlate the collected data with ICP, life of the battery, CSF flow rate, or a potential blockage in the valve assembly, and determines the ICP, the life of the battery, the CSF flow rate, or the potential blockage in the valve assembly based on the correlation and the collected data, and
the controller actuates the valve assembly based on the determination of the ICP, the life of the battery, the CSF flow rate, or the potential blockage in the valve assembly.

34. The system of claim 1, wherein the valve assembly further includes a one-way valve that allows CSF flow through the valve assembly from the intracranial space of the patient, and blocks CSF flow through the valve assembly toward the intracranial space of the patient.

35. A method for treating hydrocephalus in a patient, the method comprising:
conveying cerebrospinal fluid (CSF) from an intracranial space of the patient to a valve assembly through a proximal catheter;
generating internal sensor data with one or more sensors in the valve assembly, wherein the internal sensor data indicates an intracranial pressure (ICP) of the patient;
transmitting atmospheric pressure data from an external device or a remote server to the valve assembly, and transmitting operational data from the valve assembly to the external device;
deriving a gauge ICP based on the internal sensor data and the atmospheric pressure data collected at the valve assembly; and
actuating the valve assembly between an open position and a closed position based on the gauge ICP; or
generating an audible or visual warning, alarm, or notification with the external device when the external device attempts and fails to wirelessly communicate the atmospheric pressure data to the valve assembly a predetermined number of times or over a predetermined duration; or
generating the warning, alarm, or notification when an amount of charge in a battery operatively connected to the valve assembly or the external device reduces below a predetermined threshold, wherein the battery is operatively connected to the valve assembly, and the method further comprises:
generating the warning, alarm, or notification including an audible sound having a first intensity when the amount of charge in the battery is below a first predetermined threshold;
generating the warning. alarm, or notification including an audible sound having a second intensity greater than the first intensity when the amount of charge in the battery is below a second predetermined threshold lower than the first predetermined threshold; and
generating the warning, alarm, or notification including a visible light when the amount of charge in the battery is below a third predetermined threshold lower than the second predetermined threshold.

36. (canceled)

37. (canceled)
